(19) United States
(12) Patent Application Publication
(10) Pub. No.: US 2020/0031920 A1
(43) Pub. Date: Jan. 30, 2020

(54) Combination Therapy for Treating Cancer

(71) Applicants: The George Washington University, Washington, DC (US); The United States of America, as represented by the Secretary, Department of Health and Human Servic, Bethesda, MD (US)

(72) Inventors: Wenge ZHU, Germantown, MD (US); Wei ZHOU, Arlington, VA (US); Wei ZHENG, Potomac, MD (US); Wei SUN, Germantown, MD (US)

(21) Appl. No.: 16/500,959

(22) PCT Filed: Apr. 4, 2018
(86) PCT No.: PCT/US2018/026106

§ 371 Date: Oct. 4, 2019

Related U.S. Application Data
(60) Provisional application No. 62/481,275, filed on Apr. 4, 2017.

(51) Int. Cl. C07K 16/24; C07K 16/28; A61K 31/5025; A61K 31/506; A61K 31/5377; A61K 31/4164; A61K 31/555

(52) U.S. Cl. CPC C07K 16/244; C07K 16/2866; A61K 31/5025; A61K 45/06; A61K 31/5377; A61K 31/4164; A61K 31/555; A61K 31/506

(57) ABSTRACT

The present disclosure provides methods, pharmaceutical compositions, dosing regimens, and kits comprising a DNA damaging agent and an inhibitor of the Janus kinase 2-signal transducer and activator of transcription 5 (JAK2-STAT5) pathway, including methods of inhibiting the JAK2-STAT5 pathway in a cell, methods of treating cancer in a subject, and methods of decreasing or reversing resistance to a DNA damaging agent in a subject.

### STATEMENT OF GOVERNMENTAL INTEREST

This invention was made with government support under R01 CA184717 awarded by the National Institutes of Health. The U.S. government has certain rights in the invention.

### BACKGROUND OF THE INVENTION

Drug resistance is an obstacle that jeopardizes the efficacy of chemotherapy and reduces the overall survival rate of cancer patients. During chemotherapy, cancer cells can develop resistance to chemotherapeutic agents by adjusting their pathological signaling and gene regulatory mechanisms. Recently, cancer genome sequencing has emerged as a powerful approach to identify pathways contributing to drug resistance. However, this approach has its own limitation. For instance, it is difficult to identify target pathway(s) from sequencing data, and some unique regulatory pathways, due to post-transcriptional modification, cannot be identified by genomic sequencing.

In 2010, the U.S. Food and Drug Administration published guidance to promote development of novel combination therapies at an earlier stage of clinical development, which requires innovative technologies such as high-throughput combinational screening (HTCS) to discover the novel drug combinations. Although it is a powerful approach to identify “new” drugs that overcome resistance, HTCS has limitations with respect to identifying drug resistant mechanisms.

Janus kinases (JAKs) are part of the cytokine signal transduction pathway seen in lymphocyte development, proliferation, differentiation, and the immune response in both viral and bacterial infections during acute and chronic inflammation. The JAK/STAT pathway is also needed for embryogenesis. JAKs are recruited to cellular membrane and activated by cytokine-activated receptors. Quintas-Cardama et al., Nat Rev Drug Discov 10: 127-140 (2011). Activated JAKs phosphorylate and activate signal transducer and activator of transcription (STAT) factors, which then translocate to the nucleus to regulate the expression of genes involved in cell proliferation and apoptosis. Quintas-Cardama et al., Clin Cancer Res 19: 1933-1940 (2013). JAK2 specifically plays a role in inflammation, a hallmark of cancer. Interleukin-11 (IL-11), a member of the GP130 family, is able to signal the JAK2 pathway and activate the STAT pathway. Buchert, et al., Oncogene 35: 939-951 (2016), Bromberg, J Clin Invest 109: 1139-1142 (2002), and Ernst et al., Clin Cancer Res 20: 5579-5588 (2014). IL-11, which binds to trans-membrane IL-11 R-α, is over expressed in lung cancer, colorectal cancer, gastric cancer, breast cancer, prostate cancer, and osteosarcoma, and linked to inflammation and cancer. Id. However, the role of IL-11 in the response of cancer cells to chemotherapy remains largely unknown.

Ovarian cancer is the fifth leading cause of cancer-related deaths among women and the deadliest gynecological cancer in the United States. Siegel et al., CA Cancer J Clin 67: 7-30 (2017). The difficulty of treating ovarian cancers is underscored by the fact that ovarian cancers are genetically heterogeneous and there are no easily identifiable driver gene mutations that could be targeted for the development of therapies for a significant number of ovarian cancer patients. The current standard treatment for ovarian cancer consists of surgery followed by platinum-paclitaxel based chemotherapy. Kelland, Nat Rev Cancer 7: 573-584 (2007) and McGuire et al., N Eng J Med 334: 1-6 (1996). Platinum drugs act by entering the nucleus of the cell and forming covalent adducts with DNA, thus decreasing cell viability. Dasari et al., Eur J Pharmacol 740: 364-378 (2014). In addition to nuclear DNA damage, cisplatin can induce reactive oxygen species (ROS) response that can significantly enhance the cytotoxic effect. Choi et al., PLoS One 10: e0135083 (2015) and Marullo et al., PLoS One 8: e81162 (2013). It appears that up to 80% of patients with ovarian cancers initially respond to cisplatin-based chemotherapy and achieve remission. Armstrong et al., N Eng J Med 354: 34-43 (2006) and Burger et al., N Eng J Med 365: 2473-2483 (2011). However, cancer relapse occurs in most patients and the relapsed ovarian cancers are mostly resistant to platinum-based therapy. Hanker et al., Ann Oncol 23: 2605-2612 (2012). Studies in the past established that there are a plethora of mechanisms for cisplatin resistance, including reduced intracellular cisplatin accumulation, increased metabolic inactivation of cisplatin, increased repair of cisplatin-induced DNA damage in cells, increased tolerance of cells to the presence of cisplatin-induced DNA damage, increased anti-apoptosis capability of cells, and inactivation of p53. Galluzzi et al., Oncogene 31: 1869-1883 (2012) and Wang et al., Nat Rev Drug Discov 4: 307-320 (2005). Emerging observations also indicated a role of ROS in cisplatin resistance. Trachootham et al., Nat Rev Drug Discov 8: 579-591 (2009). However, the detailed molecular mechanism of how ROS contributes to platinum drug resistance by regulating cell survival pathways remains largely unknown. New therapeutic approaches are needed to improve patient survival in platinum-based therapy.

### BRIEF SUMMARY OF THE INVENTION

The present disclosure is directed to a pharmaceutical composition comprising a DNA damaging agent and an inhibitor of the Janus kinase 2 (JAK2)-signal transducer and activator of transcription 5 (STAT5) pathway.

In certain embodiments, the inhibitor is selected from the group consisting of: a JAK2 inhibitor, a STAT5 inhibitor, an interleukin-11 (IL-11) inhibitor, an IL-11 receptor (IL-11R) inhibitor, a Fos-related antigen 1 (FRA1) inhibitor, a reactive oxygen species (ROS) inhibitor, a ROS scavenger, and any combination thereof.

In certain embodiments, the inhibitor is a JAK2 inhibitor selected from the group consisting of: LY2784544, TG101348, TG46, and any combination thereof.

In certain embodiments, the inhibitor is selected from the group consisting of: an anti-IL-11 monoclonal antibody, an anti-IL-11R monoclonal antibody, and any combination thereof.

In certain embodiments, the inhibitor is a ROS inhibitor, a ROS scavenger, or a combination thereof, wherein the ROS inhibitor is YCG063 and the ROS scavenger is MnTMPyp.

In certain embodiments, the DNA damaging agent is a platinum-based drug. In certain embodiments, the platinum-based drug is selected from the group consisting of: cisplatin, carboplatin, diplatinum cytostatic, iproplatin, oxaliplatin, nedaplatin, satraplatin, tetraplatin, and any combination thereof.

The present disclosure is directed to a kit comprising any of the above pharmaceutical compositions.

The present disclosure is directed to a method of inhibiting the JAK2-STAT5 pathway in a cell, comprising administering to the cell: a) an effective dose of a DNA damaging agent; and b) an effective dose of an inhibitor of the JAK2-STAT5 pathway.

The present disclosure is directed to a method of treating cancer in a subject, comprising administering to the subject: a) an effective dose of a DNA damaging agent; and b) an effective dose of an inhibitor of the JAK2-STAT5 pathway.

The present disclosure is directed to a method of decreasing resistance to a DNA damaging agent that is used in the treatment of a disease or disorder in a subject, comprising administering to the subject: a) an effective dose of a DNA damaging agent; and b) an effective dose of an inhibitor of the JAK2-STAT5 pathway. In certain embodiments, the disease or disorder is a cancer.

In certain embodiments, the DNA damaging agent in any of the above methods is administered prior to, concurrently with, or subsequent to the inhibitor.

In certain embodiments, the inhibitor in any of the above methods is selected from the group consisting of: a JAK2 inhibitor, a STAT5 inhibitor, an interleukin-11 (IL-11) inhibitor, an IL-11 receptor (IL-11R) inhibitor, a Fos-related antigen 1 (FRA1) inhibitor, a reactive oxygen species (ROS) inhibitor, a ROS scavenger, and any combination thereof.

In certain embodiments, the inhibitor in any of the above methods is a JAK2 inhibitor selected from the group consisting of: LY2784544, TG101348, TG46, and any combination thereof.

In certain embodiments, the inhibitor in any of the above methods is selected from the group consisting of: an anti-IL-11 monoclonal antibody, an anti-IL-11R monoclonal antibody, and a combination thereof.

In certain embodiments, the inhibitor in any of the above methods is a ROS inhibitor, a ROS scavenger, or a combination thereof, wherein the ROS inhibitor is YCG063 and the ROS scavenger is MnTMPyp.

In certain embodiments, prior to initiation of any of the above methods the subject has been identified as having a cancer that is resistant to treatment with at least one DNA damaging agent.

In certain embodiments, the cancer in any of the above methods is selected from the group consisting of: ovarian cancer, testicular cancer, bladder cancer, head and neck cancer, oral cancer, esophageal cancer, lung cancer, small cell lung cancer, non-small cell lung cancer, breast cancer, cervical cancer, stomach cancer, gastric cancer, colorectal cancer, osteosarcoma, pancreatic cancer, prostate cancer, and any combination thereof.

In certain embodiments, the DNA damaging agent in any of the above methods is a platinum-based drug. In certain embodiments, the platinum-based drug is selected from the group consisting of: cisplatin, carboplatin, diplatinum cytostatic, iproplatin, oxaliplatin, nedaplatin, satraplatin, tetraplatin, and any combination thereof.

In certain embodiments, prior to initiation of any of the above methods the level of IL-11 mRNA or IL-11 protein, ROS, or any combination thereof in cells or blood serum in the subject is higher than in control cells or blood serum.

### BRIEF DESCRIPTION OF DRAWINGS

FIG. 1 is a line graph showing the proliferation of SKOV3 parental and SKOV3 CR cells treated with increasing concentrations of cisplatin for five days. Data are represented as mean±SD from three independent experiments performed in triplicate. Indicated IC₅₀ values represent the mean of two independent experiments performed in triplicate.

FIG. 2 is an illustration showing representative flow plots (A) and a bar graph (B) of SKOV3 and SKOV3 CR cells treated with cisplatin for 48 h and analyzed for Annexin V and Propidium Iodide staining by flow cytometry (A) and quantified apoptosis percentage (B). Data are represented as mean±SD from three independent experiments performed in triplicate. **p<0.01.

FIG. 3 is an illustration showing the effect of increasing concentrations of cisplatin on the cleavage of caspase-9 and PARP as assessed in SKOV3 parental cells as compared with SKOV3 CR3 cells.

FIG. 4 is an illustration showing representative TUNEL staining (A) and a bar graph (B) of SKOV3 and SKOV3 CR xenograft tumors treated with cisplatin for two weeks (2 mg/kg or 4 mg/kg cisplatin twice per week) and quantified apoptosis percentage (B). n−3 mice/group. Bar in (A)−50 μm. Data are represented as mean±SD. **p<0.01, ***p<0.001.

FIG. 5 is a flow chart for HTS compound screening. The criteria for compound selection and the number of compounds at each step are listed.

FIG. 6 is an illustration showing enrichment of SKOV3 CR for a strong response to specific drug categories (rows). Drug-category-response scores are based on IC₅₀(μM).

FIG. 7 is a line graph showing the activity of the LY2784544/cisplatin combination.

FIG. 8 is a line graph showing the activity of the LY2784544/cisplatin combination.

FIG. 9 is a line graph showing the activity of the MLN4924/cisplatin combination.

FIG. 10 is a line graph showing the activity of the MLN4924/cisplatin combination.

FIG. 11 is a line graph showing the activity of the NSC319726/cisplatin combination.

FIG. 12 is a line graph showing the activity of the NSC319726/cisplatin combination.

FIG. 13 is a line graph showing the proliferation of SKOV3 parental and SKOV3 CR cells treated with increasing concentrations of cisplatin and 0.4 μM LY2784544 for five days. Data are represented as mean±SD from three independent experiments performed in triplicate.

FIG. 14 is a scatter plot showing the isobologram analysis of LY2784544 and cisplatin at multiple concentrations in SKOV3 cells. Results are from a representative experiment performed in triplicate. CI, combination index.

FIG. 15 is a scatter plot showing the isobologram analysis of LY2784544 and cisplatin at multiple concentrations in SKOV3 CR cells. Results are from a representative experiment performed in triplicate. CI, combination index.

FIG. 16 is an illustration showing representative colony formation (A) and bar graphs ((B)-(C)). SKOV3 parental and resistant cells were treated with 0.1 μM LY2784544 and 0.5 μM cisplatin for 14 days and quantification data of colony formation assays ((B) and (C)). Colonies in (A) were stained with crystal violet. Data are represented as mean±SD from three independent experiments performed in triplicate. *p<0.05, **p<0.01, ***p<0.001.

FIG. 17 is a bar graph showing synergistic effects of TG101348 and cisplatin in SKOV3 CR cells. CI values are presented above the bars. CI<1 indicates synergism, CI=1 indicates additive effect, and CI>1 indicates antagonism.

FIG. 18 is a bar graph showing synergistic effects of TG46 and cisplatin in SKOV3 CR cells. CI values are presented above the bars. CI<1 indicates synergism, CI=1 indicates additive effect, and CI>1 indicates antagonism.

FIG. 19 is an illustration showing the expression of phosphorylation of JAK2 and STAT5 in ovarian cancer parental and resistant cells.

FIG. 20 is an illustration showing that JAK2 knockdown inhibits signaling in puromycin-selected SKOV3 CR cells.

FIG. 21 is a bar graph showing the proliferation of SKOV3 CR cells treated with increasing concentrations of cisplatin for 5 days after JAK2 knockdown. Data are represented as mean±SD from three independent experiments performed in triplicate. *p<0.05, **p<0.01.

FIG. 22 is an illustration showing that LY2784544 down-regulates JAK2/STAT5 signaling in SKOV3 CR cells. Cells were treated with the indicated concentrations of LY2784544 for 48 hr.

FIG. 23 is an illustration showing the immunoblotting for the indicated targets in SKOV3 CR cells treated with vehicle (DMSO), 3 μM LY2784544, 5 μM cisplatin, or the combination for 48 hr.

FIG. 24 is an illustration showing representative flow plots of SKOV3 CR cells treated with vehicle, 3 μM LY2784544, 5 μM cisplatin, or the combination (combo) for 48 hr and analyzed for Annexin V and Propidium Iodide staining by flow cytometry.

FIG. 25 is a bar graph showing the quantified apoptosis percentage in SKOV3 CR cells. Data are represented as mean±SD from three independent experiments performed in triplicate. **p<0.01.

FIG. 26 are line graphs showing the growth curves of tumors (A) and Kaplan-Meier survival curves (C) from mice treated with vehicle, LY2784544 (15 mg/kg/day intraperitoneally), cisplatin (8 mg/kg/week intraperitoneally), or LY2784544 plus cisplatin (combo) for 2 weeks. A photograph of the representative tumor from mice in each treatment arm is also shown (B). Data in (A) are represented as mean±SEM, n=6 mice/group. *p<0.05, **p<0.01, ***p<0.001. Data in (B) are represented as n=6 mice/group. *p<0.05, **p<0.01. Ruler scale in (B) is in cm.

FIG. 27 is an illustration showing the IHC and TUNEL staining of tumors from mice in each treatment arm sacrificed after 4 days of treatment. n=3 mice/group. Bar=50 μm.

FIG. 28 is a bar graph showing quantification of apoptosis cells percentage in tumors treated as in (H). Data are represented as mean±SD. n=3 mice/group. **p<0.01, ***p<0.001.

FIG. 29 is an illustration of a heat map diagram with genes over 2-fold up and down regulated in SKOV3 compared with SKOV3 cells.

FIG. 30 is an illustration of a heat map diagram with JAK2 related cytokine gene in SKOV3 and SKOV3 CR cells.

FIG. 31 is an illustration of cytokine arrays showing expression of the indicated cytokines in supernatants of SKOV3 and SKOV3 CR cells (A) and PEO1 and PEO4 cells (B).

FIG. 32 is a bar graph showing the proliferation of SKOV3 parental cells treated with increasing concentrations of cisplatin for 5 days after being pretreated for 48 hr with conditioned medium. Data are represented as mean±SD from three independent experiments performed in triplicate. *p<0.05, **p<0.01, ***p<0.001.

FIG. 33 is an illustration showing the expression of phosphorylation of JAK2 and STAT5 in SKOV3 parental cells after treatment for 48 hr with conditioned media from SKOV3 parental and CR cells.

FIG. 34 is a series of three bar graphs ((A)-(C)) showing the levels of mRNA in ovarian cancer parental and resistant cell lines. Levels of IL-11 were measured by ELISA and RT-qPCR and are shown as mean±SD from three independent experiments performed in triplicate. **p<0.01.

FIG. 35 is a series of three bar graphs ((A)-(C)) showing the levels of secreted IL-11 in ovarian cancer parental and resistant cell lines. Levels of IL-11 were measured by ELISA and RT-qPCR and are shown as mean±SD from three independent experiments performed in triplicate. **p<0.01.

FIG. 36 is an illustration showing representative H&E and IHC images of SKOV3 parental and SKOV3 CR cells xenograft tumor. Bar=50 μm.

FIG. 37 is an illustration showing the phosphorylation of JAK2 and STAT5 in SKOV3 parental cells treated with 10 ng/mL of IL-11 for 4 hr by western blot analysis.

FIG. 38 is a bar graph showing the proliferation of SKOV3 parental cells incubated with IL-11 for 4 hr and then treated with increasing concentrations of cisplatin for 5 days. Data are represented as mean±SD from three independent experiments performed in triplicate. *p<0.05, **p<0.01.

FIG. 39 is an illustration showing the phosphorylation of JAK2 and STAT5 in SKOV3 CR and IGROV1 CR cells treated with neutralizing IL-11 Ab for 4 hours.

FIG. 40 is a bar graph showing the proliferation of SKOV3 CR cells incubated with neutralizing IL-11 Ab for 4 hr and then treated with increasing concentrations of cisplatin for 5 days. Data are represented as mean±SD from three independent experiments performed in triplicate. *p<0.05, **p<0.01, ***p<0.001.

FIG. 41 is a bar graph showing the proliferation of IGROV1 CR cells incubated with neutralizing IL-11 Ab for 4 hr and then treated with increasing concentrations of cisplatin for 5 days. Data are represented as mean±SD from three independent experiments performed in triplicate. *p<0.05, **p<0.01, ***p<0.001.

FIG. 42 is a bar graph showing IL-11 knockdown inhibits secreted IL-11 in puromycin-selected SKOV3 CR cells.

FIG. 43 is an illustration showing IL-11 knockdown inhibits JAK2 signaling in puromycin-selected SKOV3 CR cells.

FIG. 44 is a bar graph showing the proliferation of SKOV3 CR cells treated with increasing concentrations of cisplatin for 5 days after the IL11 knockdown. Data are represented as mean±SD from three independent experiments performed in triplicate. *p<0.05, **p<0.01, ***p<0.001.

FIG. 45 is a bar graph (A) and line graph (B), showing that recombinant human IL-11 reversed endogenous IL11 knockdown mediated sensitivity of SKOV3 CR cells to cisplatin (A) but could not reverse endogenous JAK2 knockdown mediated sensitivity. Data in (A) are represented as mean±SD from three independent experiments performed in triplicate. *p<0.05, **p<0.01. For (B), ***p<0.001.

FIG. 46 is a bar graph showing levels of IL-11 measured by ELISA in SKOV3 parental cells treated with cisplatin at various dosages. Data are represented as mean±SD from three independent experiments performed in triplicate. *p<0.05, **p<0.01, ***p<0.001.

FIG. 47 is a bar graph showing levels of IL-11 measured by ELISA in SKOV3 parental cells treated with cisplatin for various times. Data are represented as mean±SD from three independent experiments performed in triplicate. *p<0.05, **p<0.01, ***p<0.001.

FIG. 48 is a bar graph showing levels of IL-11 measured by qPCR in SKOV3 parental cells treated with cisplatin at various dosages. Data are represented as mean±SD from three independent experiments performed in triplicate. *p<0.05, **p<0.01, ***p<0.001.

FIG. 49 is a bar graph showing levels of IL-11 measured by qPCR in SKOV3 parental cells treated with cisplatin for various times. Data are represented as mean±SD from three independent experiments performed in triplicate. *p<0.05, **p<0.01, ***p<0.001.

FIG. 50 is an illustration showing the expression of phosphorylation of JAK2 and STAT5 in SKOV3 parental cells treated with cisplatin at various dosages.

FIG. 51 is an illustration showing the expression of phosphorylation of JAK2 and STAT5 in SKOV3 parental cells treated with cisplatin at various times (days).

FIG. 52 is a bar graph showing IL-11 levels measured by ELISA in plasma of mice bearing SKOV3 xenograft tumors treated with vehicle or cisplatin (2-6 mg/kg/twice a week intraperitoneally for 2 weeks). Data are represented as mean±SD. n=3 mice/group. **p<0.01, ***p<0.001.

FIG. 53 is an illustration showing representative H&E and IHC images of SKOV3 parental xenograft tumors.

FIG. 54 are line graphs ((A) and C)) and bar graphs ((B) and (D)) showing that expression of IL11 gene mRNAs in ovarian cancer predicts clinical outcome via Kaplan-Meier analyses of 5-year progression-free survival and overall survival. P values were determined by log-rank test.

FIG. 55 are line graphs ((A) and B)) showing that expression of JAK2 signature genes in ovarian cancer predicts clinical outcome via Kaplan-Meier analyses of 5-year progression-free survival and overall survival. P values were determined by log-rank test.

FIG. 56 is a line graph showing the proliferation of SKOV3 parental and SKOV3 CR cells treated with increasing concentrations of carboplatin for 5 days. Data are represented as mean±SD.

FIG. 57 is a bar graph showing the synergistic effects of LY2784544 and carboplatin in SKOV3 CR cells. CI values are presented above the bars. CI<1 indicates synergism, CI=1 indicates additive effect, and CI>1 indicates antagonism.

FIG. 58 is a line graph showing the relative cell viability of SKOV3 CR cells treated with increasing concentrations of cisplatin and 0.1 μM/0.2 μM MLN4924 for five days. Data are represented as mean±SD from three independent experiments performed in triplicate.

FIG. 59 is a scatter plot showing isobologram analysis of MLN4924 and cisplatin at multiple concentrations in SKOV3 CR cells.

FIG. 60 is a line graph showing the proliferation of IGROV1 parental and IGROV1 CR cells treated with increasing concentrations of cisplatin for 5 days (left). Data are represented as mean±SD from three independent experiments performed in triplicate. Indicated IC₅₀ values represent the mean of two independent experiments performed in triplicate.

FIG. 61 is a bar graph showing the synergistic effects of LY2784544 and cisplatin in IGROV1 CR cells (right). CI values are presented above the bars. CI<1 indicates synergism, CI−1 indicates additive effect, and CI>1 indicates antagonism.

FIG. 62 is an illustration showing PEO1 and PEO4 established at different time points through disease progression.

FIG. 63 is a line graph showing the proliferation of PEO1 and PEO4 cells treated with increasing concentrations of cisplatin for 5 days. Data are represented as mean±SD from three independent experiments performed in triplicate. Indicated IC₅₀ values represent the mean of two independent experiments performed in triplicate.

FIG. 64 is a bar graph showing the synergistic effects of LY2784544 and cisplatin in PEO4 cells. CI values are presented above the bars. CI<1 indicates synergism, CI=1 indicates additive effect, and CI>1 indicates antagonism.

FIG. 65 is a line graph showing body weights of tumor-bearing mice during treatment as in FIG. 26. Data are represented as mean±SD, n=6 mice/group.

FIG. 66 is an illustration showing the histopathology of liver and kidney collected from mice 4 days after the final treatment as in FIG. 26. Bar=50 μm.

FIG. 67 is an illustration showing the heat map diagram with a JAK2-related receptor gene in SKOV3 CR compared with SKOV3 cells from RNA sequencing.

FIG. 68 is an illustration showing the expression of JAK2-related receptor gene in SKOV3 CR compared with SKOV3 assessed by western blot.

FIG. 69 are bar graphs ((A)-(C) and (E)-(F)) and illustrations ((D) and (G)) showing that reactive oxygen species (ROS) induces IL-11 expression. (A) is a bar graph showing quantification of ROS production in SKOV3 and SKOV3 CR cells. Data are represented as means±SD from three independent experiments. **p<0.01. Scale bar, 100 μm. (B) is a bar graph showing quantification of ROS production in SKOV3 CR cells after treatment with the ROS inhibitor YCG063 at 20 μM for 24 hr. Data are represented as means±SD from three independent experiments. ***p<0.001. (C) is a bar graph showing IL-11 levels measured by ELISA in the medium of SKOV3 CR cells treated with YCG063 at 20 μM for 24 hr. Data are represented as mean±SD from three independent experiments performed in triplicate. ***p<0.001. (D) is an illustration showing an immunoblot of phosphorylated JAK2 and STAT5 in SKOV3 CR cells treated with YCG063 (20 μM) for 24 hr. (E) is a bar graph showing quantification of ROS production in SKOV3 cells after treated with the ROS inhibitor YCG063 (20 μM), cisplatin (1 μM), or both for 24 hr. Data are represented as means±SD from three independent experiments. **p<0.01, ***p<0.001, n.s. means not significant by one-way ANOVA. (F) is a bar graph showing IL-11 levels measured by ELISA in SKOV3 cells treated with YCG063 (20 μM), cisplatin (1 μM) or both for 24 hr. Data are represented as mean±SD from three independent experiments performed in triplicate. ***p<0.001 by one-way ANOVA. (G) is is an illustration showing an immunoblot of phosphorylated JAK2 and STAT5 in SKOV3 CR cells treated with YCG063 (20 μM), cisplatin (1 μM) or both for 24 hr.

FIG. 70 are illustrations ((A), (B), and (D)) and a bar graph (C) showing that ROS induces IL-11 expression by promoting expression of FOSL1 (FRA1). (A) is an illustration showing total and phosphorylated levels of FRA1 in SKOV3 and SKOV3 CR cells. (B) shows that depletion of FOSL1 by siRNA decreases the phosphorylated and total FRA1 protein levels in SKOV3 CR cells. (C) is a bar graph showing IL-11 levels measured by ELISA in the medium of SKOV3 CR cells transfected with FOSL1 siRNA for 48 hr. Data are represented as mean±SD from three independent experiments performed in triplicate. ***p<0.001. (D) is an illustration showing an immunoblot of phosphorylated and total FRA1 protein in SKOV3 CR cells treated with YCG063 20 μM for 24 hr.

FIG. 71 are graphs ((A)-(C)) showing decreased survival in ovarian cancer patients with higher IL-11 mRNA levels. (A) is a graph showing a comparison of the mRNA IL-11 levels measured by qPCR in platinum sensitive (n=23) and resistant (n=16) ovarian cancer patients. *p<0.05. Sen, sensitive cases. Res, resistant cases. (B) and (C) are line graphs showing Kaplan-Meier survival curves showing 5-year PFS rate (B) and OS rate (C) of 39 ovarian cancer patients stratified by IL11 mRNA levels by median cutoff; log-rank (Mantel-Cox), P values and HRs are shown.

FIG. 72 are graphs ((A)-(C) and (E)) and a bar graph (D) showing decreased survival in ovarian cancer patients with higher serum IL-11 levels ((A)-(C)) and activated IL-11-JAK2 pathway in patients. (A) shows a comparison of serum IL-11 levels measured by ELISA in platinum sensitive (n=21) and resistant (n=16) ovarian cancer patients. **p<0.01. Sen, sensitive cases. Res, resistant cases. (B) and (C) show Kaplan-Meier survival curves showing 5-year PFS rate (B) and OS rate (C) of 37 ovarian cancer patients were stratified by serum IL-11 levels (40 pg/ml); logrank (Mantel-Cox), P values and HRs are shown. (D) shows quantification of IL-11 and JAK2 levels as determined by immunohistochemistry for cisplatin sensitive and resistant tumor samples from the same patient. (E) shows scatter plots showing a correlation between IL-11 and pJAK2 levels in both primary and recurrent patient tumors.

### DETAILED DESCRIPTION OF THE INVENTION

The present disclosure provides methods, pharmaceutical compositions, dosing regimens, and kits comprising a DNA damaging agent and an inhibitor of the Janus kinase 2 (JAK2)-signal transducer and activator of transcription 5 (STAT5) pathway, including methods of inhibiting the JAK2-STAT5 pathway in a cell, methods of treating cancer in a subject and methods of decreasing or reversing resistance to a DNA damaging agent in a subject.

The headings provided herein are not limitations of the various aspects or aspects of the disclosure, which can be defined by reference to the specification as a whole. Accordingly, the terms defined immediately below are more fully defined by reference to the specification in its entirety. Before describing the present disclosure in detail, it is to be understood that this invention is not limited to specific compositions or process steps, as such can vary.

### I. Terminology

Unless defined otherwise, all technical and scientific terms used herein have the same meaning as commonly understood by one of ordinary skill in the art to which this invention belongs. In case of conflict, the present application including the definitions will control. Unless otherwise required by context, singular terms shall include pluralities and plural terms shall include the singular.

As used herein and in the appended claims, the singular forms “a,” “an,” and “the” include plural references unless the context clearly dictates otherwise. For example, the term “an inhibitor” or “at least one inhibitor” can include a plurality of inhibitors, including mixtures thereof. The terms “a”, “an,” “the,” “one or more,” and “at least one,” for example, can be used interchangeably herein.

As used herein, the term “about,” when used to modify an amount related to the invention, refers to variation in the numerical quantity that can occur, for example, through routine testing and handling; through inadvertent error in such testing and handling; through differences in the manufacture, source, or purity of ingredients employed in the invention; and the like. Whether or not modified by the term “about”, the claims include equivalents of the recited quantities. In some embodiments, the term “about” means plus or minus 10% of the reported numerical value.

Throughout this application, various embodiments of this invention can be presented in a range format. It should be understood that the description in range format is merely for convenience and brevity and should not be construed as an inflexible limitation on the scope of the invention. Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. Accordingly, the description of a range should be considered to have specifically disclosed all the possible subranges as well as individual numerical values within that range. For example, description of a range, such as from 1 to 6 should be considered to have specifically disclosed subranges such as from 1 to 2, from 1 to 3, from 1 to 4, from 1 to 5, from 2 to 3, from 2 to 4, from 2 to 5, from 2 to 6, from 3 to 4, from 3 to 5, from 3 to 6, etc., as well as individual numbers within that range, for example, 1, 2, 3, 4, 5, and 6, and subranges of less than whole number such as 1.1, 1.2, 1.3, 1.4, etc. This applies regardless of the breadth of the range.

The terms “comprises,” “comprising,” “includes,” “including,” “having,” and their conjugates are interchangeable and mean “including but not limited to.” It is understood that wherever aspects are described herein with the language “comprising,” otherwise analogous aspects described in terms of “consisting of” and/or “consisting essentially of” are also provided.

The term “consisting of” means “including and limited to.”

The term “consisting essentially of” means the specified material of a composition, or the specified steps of a method, and those additional materials or steps that do not materially affect the basic characteristics of the material or method.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the term “effective dose” of an agent is that amount sufficient to effect beneficial or desired results, for example, clinical results, and, as such, an “effective dose” depends upon the context in which it is being applied. The term “effective dose” can be used interchangeably with “effective amount,” “therapeutically effective amount,” “therapeutically effective dose,” “clinically effective amount,” or “clinicially effective dose.”

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Administration of any one agent as described herein “in combination with” one or more other agents includes simultaneous (concurrent) and consecutive administration in any order. By “combination” or “in combination with,” it is not intended to imply that the therapy or the therapeutic agents must be administered at the same time and/or formulated for delivery together (e.g., in the same composition), although these methods of delivery are within the scope described herein.

The terms “invention” and “disclosure” can be used interchangeably when describing or used, for example, in the phrases “the present invention” or “the present disclosure.”

As used herein, the terms “chemotherapeutic agent” and “chemotherapeutic drug” are interchangeable and refer to a chemical compound useful in the treatment of cancer, regardless of mechanism of action.

As used herein, the term “excipient” refers to a component, or mixture of components, that is used to give desirable characteristics to a pharmaceutical composition or dosage form as disclosed herein. An excipient of the present invention can be described as a “pharmaceutically acceptable” excipient, meaning that the excipient is a compound, material, composition, salt, and/or dosage form which is, within the scope of sound medical judgment, suitable for contact with tissues of animals (i.e., humans and non-human animals) without excessive toxicity, irritation, allergic response, or other problematic complications over the desired duration of contact commensurate with a reasonable benefit/risk ratio.

As used herein, the term “expression” when used in relation to a nucleic acid refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

As used herein, the term “pharmaceutical composition” refers to a preparation which is in such form as to permit the biological activity of the active ingredient to be effective, and which contains no additional components which are unacceptably toxic to a subject to which the composition would be administered. Such composition can be sterile.

As used herein, the term “subject” or “individual” or “animal” or “patient” or “mammal,” means any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; bears, food animals such as cows, pigs, and sheep; ungulates such as deer and giraffes; rodents such as mice, rats, hamsters and guinea pigs; and so on. In certain embodiments, the mammal is a human subject. In other embodiments, a subject is a human patient. In certain embodiments, a subject is a human patient in need of a cancer treatment. In certain embodiments, a subject is a human male and/or a human female. The term “cancer patient” as used herein is meant to include any subject being treated for cancer, including, but not limited to, humans and veterinary animals.

As used herein, the term “treating” or “treatment” or “therapy” refers to partially or completely alleviating, ameliorating, improving, relieving, delaying onset of, inhibiting progression of, reducing severity of, and/or reducing incidence of one or more symptoms or features of disease or disorder, including a condition, (e.g., a cancer). For example, “treating” a cancer can refer to inhibiting growth and/or spread of a cancer. Treatment can be administered to a subject who does not exhibit signs of a disease or disorder and/or to a subject who exhibits only early signs of a disease or disorder for the purpose of decreasing the risk of developing pathology associated with the disease or disorder.

It is appreciated that certain features of the invention, which are, for clarity, described in the context of separate embodiments, can also be provided in combination in a single embodiment. Conversely, various features of the invention, which are, for brevity, described in the context of a single embodiment, can also be provided separately or in any suitable subcombination or as suitable in any other described embodiment of the invention. Certain features described in the context of various embodiments are not to be considered essential features of those embodiments, unless the embodiment is inoperative without those elements.

Although methods and materials similar or equivalent to those described herein can be used in practice or testing of the present invention, suitable methods and materials are described below. The materials, methods and examples are illustrative only and are not intended to be limiting. Other features and advantages of the invention will be apparent from the detailed description and from the claims.

### II. Pharmaceutical Compositions and Kits

In one aspect, the present invention is directed to a pharmaceutical composition comprising a DNA damaging agent and an inhibitor of the Janus kinase 2 (JAK2)-signal transducer and activator of transcription 5 (STAT5) pathway.

In another aspect, the present invention is directed to a dosing regimen comprising a DNA damaging agent and an inhibitor of the JAK2-STAT5 pathway. In some embodiments, the dosing regimen comprises a dosage form comprising the DNA damaging agent and the inhibitor. In some embodiments, the dosing regimen comprises a first dosage form comprising the DNA damaging agent and a second dosage form comprising the inhibitor. In some embodiments, the first dosage form is for administration prior to, concurrently with, or subsequent to the second dosage form.

In another aspect, a pharmaceutical composition or a dosing regimen as disclosed herein is for use in inhibiting the JAK2-STAT5 pathway in a cell. In some embodiments, the cell is in vitro. In some embodiments, the cell is in vivo (e.g., in a subject).

In another aspect, a pharmaceutical composition or a dosing regimen as disclosed herein is for use in treating cancer.

In another aspect, a pharmaceutical composition or a dosing regimen as disclosed herein is for decreasing resistance to a DNA damaging agent that is used in the treatment of a disease or disorder in a subject. The term “resistance to a DNA damaging agent” can be used interchangeably with the term “tolerance to a DNA damaging agent” and refers to a diminishing therapeutic benefit of a DNA damaging agent in treating a disease or disorder in a subject over time. “Decreasing” resistance or tolerance as referred to herein can include any decrease in resistance or tolerance that provides a therapeutic benefit, including preventing or delaying development of resistance or tolerance in a subject or reducing or eliminating an existing resistance or tolerance in a subject. In some embodiments, a pharmaceutical composition or a dosing regimen as disclosed herein is for preventing or delaying development of resistance or tolerance to a DNA damaging agent in a subject. In some embodiments, a pharmaceutical composition or a dosing regimen as disclosed herein is for reducing or eliminating an existing resistance or tolerance to a DNA damaging agent in a subject. In some embodiments, a pharmaceutical composition or a dosing regimen as disclosed herein is for treating a disease or disorder in a subject with existing resistance or tolerance to a DNA damaging agent. In some embodiments, the disease or disorder is cancer.

In some embodiments, the cancer is selected from the group consisting of: ovarian cancer, testicular cancer, bladder cancer, head and neck cancer, oral cancer, esophageal cancer, lung cancer, small cell lung cancer, non-small cell lung cancer, breast cancer, cervical cancer, stomach cancer, gastric cancer, colorectal cancer, osteosarcoma, pancreatic cancer, prostate cancer, and any combination thereof. In some embodiments, the cancer is ovarian cancer.

A “DNA damaging agent” can be any therapeutic agent that causes DNA damage, including, but not limited to: chemotherapeutic agents, DNA alkylating agents, nucleoside analogs, replication inhibitors, platinum-based drugs, actinomycin, amsacrine, cyclophosphamide (Cytoxan®), dactinomycin, daunorubicin, doxorubicin, epirubicin, iphosphamide, merchlorehtamine, mitomycin, mitoxantrone, nitrosourea, procarbazine, taxol, taxotere, teniposide, etoposide, triethylenethiophosphoramide, hydroxyurea, gemcitabine, or any combination thereof.

In some embodiments, the DNA damaging agent is a DNA alkylating agent, including, but not limited to: mechlorethamine, uramustine, streptozocin, busulfan, Shionogi 254-S, aldo-phosphamide analogues, altretamine, anaxirone, Boehringer Mannheim BBR-2207, bendamustine, bestrabucil, budotitane, Wakunaga CA-102, carmustine, Chinoin-139, Chinoin-153, cyclophosphamide, American Cyanamid CL-286558, Sanofi CY-233, cyplatate, Degussa D-19-384, Sumimoto DACHP(Myr)2, diphenylspiromustine, diplatinum cytostatic, Erba distamycin derivatives, Chugai DWA-2114R, ITI E09, elmustine, Erbamont FCE-24517, estramustine phosphate sodium, fotemustine, Unimed G-6-M, Chinoin GYKI-17230, hepsul-fam, ifosfamide, iproplatin, lomustine, mafosfamide, melphalan, mitolactol, Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, Yakult Honsha SN-22, spiromustine, Tanabe Seiyaku TA-077, tauromustine, temozolomide, teroxirone, tetraplatin, trimelamol, or any combination thereof.

In some embodiments, the DNA damaging agent is a platinum-based drug, including a platinum analog or platinum. The terms “platinum-based drug” and “platinum-based chemotherapeutic drug” can be used interchangeably herein. In some embodiments, the platinum-based drug includes, but is not limited to, cisplatin, carboplatin, diplatinum cytostatic, iproplatin, oxaliplatin, nedaplatin, satraplatin, tetraplatin, or any combination thereof.

An inhibitor of the JAK2-STAT5 pathway can be any one or more agents that inhibits or reduces, including eliminates, substantially eliminates, or prevents, a JAK2 and/or STAT5 activity, activation of JAK2 and/or STAT5, and/or expression of JAK2 and/or STAT5. In some embodiments, the inhibitor inhibits or reduces, including eliminates, substantially eliminates, or prevents, the phosphorylation of JAK2 and/or STAT5. In some embodiments, the inhibitor inhibits or reduces, including eliminates, substantially eliminates, or prevents, the phosphorylation of tyrosine residue 1007 and/or 1008 of human JAK2, and/or phosphorylation of tyrosine residue 694 of human STAT5. In some embodiments, an inhibitor of the JAK2-STAT5 pathway inhibits or reduces, including eliminates, substantially eliminates, or prevents, an activity, activation, or expression of an upstream member of the JAK2-STAT5, resulting in inhibition of JAK2 and/or STAT5. In some embodiments, the upstream member of the JAK2-STAT5 pathway is selected from the group consisting of interleukin-11 (IL-11), IL-11 receptor (IL-11R), Fos-related antigen 1 (FRA1), a reactive oxygen species (ROS), a ROS scavenger, and any combination thereof.

In some embodiments, the inhibitor is a small molecule, an antibody, or an oligonucleotide.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, Fv, Fsc, CDR regions, or any portion of an antibody that is capable of binding an antigen or epitope), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. The modifier “monoclonal” indicates the character of the antibody as being obtained from a substantially homogeneous population of antibodies, and is not to be construed as requiring production of the antibody by any particular method. The term “antibody” as used herein also includes single-domain antibodies (sdAb) and fragments thereof that have a single monomeric variable antibody domain (VH) of a heavy-chain antibody. sdAb, which lack variable light (VL) and constant light (CL) chain domains are natively found in camelids (VHH) and cartilaginous fish (VNAR) and are sometimes referred to as “Nanobodies” by the pharmaceutical company Ablynx who originally developed specific antigen binding sdAb in llamas. An antibody can be any of the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc. (e.g., immunoconjugates).

In some embodiments, the antibody is a blocking antibody or antagonist antibody. A “blocking” antibody or an “antagonist” antibody is one which inhibits or reduces biological activity of the antigen it binds. In some embodiments, blocking antibodies or antagonist antibodies substantially or completely inhibit the biological activity of the antigen. The biological activity can be reduced, for example, by about 10%, about 20%, about 30%, about 40%, about 50%, about 60%, about 70%, about 80%, about 90%, about 95%, or about 100%.

In some embodiments, the antibody is an “antibody fragment,” which refers to an antigen-binding portion of an intact antibody. Examples of antibody fragments include, but are not limited to Fab, Fab′, F(ab′)2, and FAT fragments, linear antibodies, single chain antibodies, and multispecific antibodies formed from antibody fragments.

In some embodiments, the antibody specifically binds a target (e.g, specifically binds FRA1, IL-11, JAK2, or STAT5). By “specifically binds,” it is generally meant that an antibody binds to an epitope of a target via the antibody's antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope.

An oligonucleotide inhibitor can include RNA and/or DNA, and modified forms thereof, capable of binding to a target nucleic acid and preventing expression of the target nucleic acid, including, but not limited to, antisense DNA/RNA, small interfering (siRNA), microRNA (miRNA), asymmetrical interfering RNA (aiRNA), Dicer-substrate RNA (dsRNA), and small hairpin RNA (shRNA).

In some embodiments, an inhibitor of the JAK2-STAT5 pathway as disclosed herein is selected from the group consisting of: a JAK2 inhibitor, a STAT5 inhibitor, an interleukin-11 (IL-11) inhibitor, an IL-11 receptor (IL-11R) inhibitor, a Fos-related antigen 1 (FRA1) inhibitor, a reactive oxygen species (ROS) inhibitor, a ROS scavenger, and any combination thereof.

In some embodiments, a JAK2 inhibitor as disclosed herein includes, but is not limited to, an anti-JAK2 antibody (such as, for example, Ruxolitinib, Baricitinib, Filgotinib, Gandotinib, Lestaurtinib, Momelotinib, Pacrinitib, CHZ868, Fedratinib, Cucurbitacin I, or any combination thereof), an oligonucleotide, LY2784544 (i.e., Gandotinib), TG101348 (Fedratinib), TG46, or any combination thereof.

In some embodiments, the JAK2 inhibitor is LY2784544, which has the following Structure I:

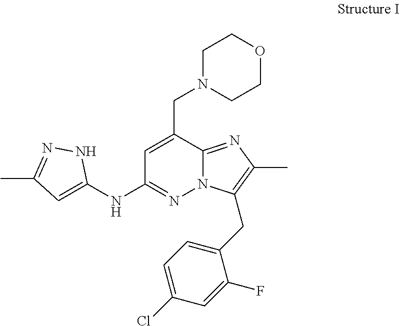

In some embodiments, the JAK2 inhibitor is TG101348, which has the following Structure II:

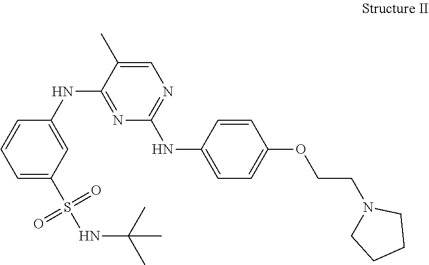 [SMILES: Cc1cnc(Nc2ccc(OCCN3CCCC3)cc2)nc1Nc1cccc(S(=O)(=O)NC(C)(C)C)c1]

In some embodiments, the JAK2 inhibitor is TG46, which has the following Structure III:

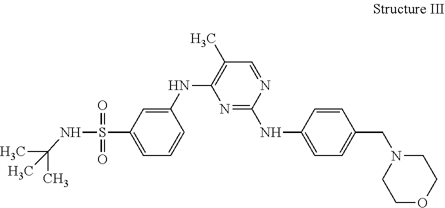 [SMILES: Cc1cnc(Nc2ccc(CN3CCOCC3)cc2)nc1Nc1cccc(S(=O)(=O)NC(C)(C)C)c1]

In some embodiments, the inhibitor is a STAT5 inhibitor. STAT5 is known to be activated by JAK2 and is therefore responsible for cell signaling downstream from JAK2. Ma et al., Blood Cancer J 3: e109 (2013) and Wu et al., Cancer Cell 28: 29-41 (2015). In some embodiments, a STAT5 inhibitor as disclosed herein includes, but is not limited to, an anti-STAT5 antibody, an oligonucleotide, pimozide (Structure IV), N′-((4-Oxo-4 H-chromen-3-yl)methylene)nicotinohydrazide (also termed 2-[(4-oxo-4H-1-benzopyran-3-yl) methylene]hydrazide 3-pyridinecarboxylic acid) (Structure V), Structure VI, BP-1-108, SF-1-088, and any combination thereof. BP-1-108 and SF-1-088 are disclosed in Cumaraswamy et al., ACS Med Chem Lett. 5:1202-1206 (2014).

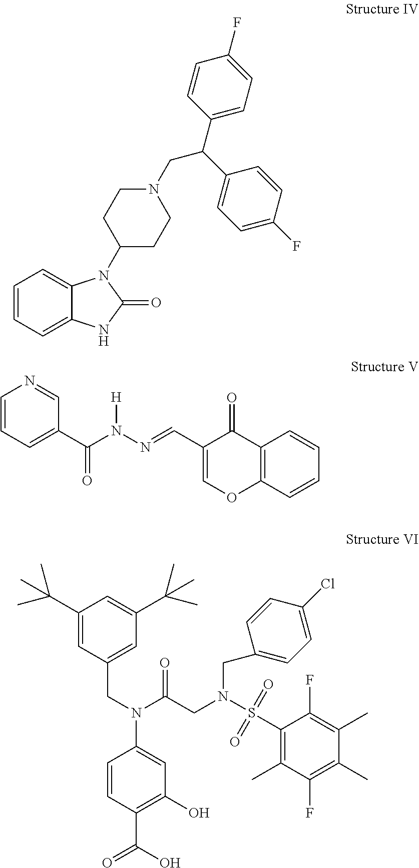

In some embodiments, an IL-11 inhibitor as disclosed herein includes, but is not limited to, an anti-IL-11 monoclonal antibody, an oligonucleotide, or a combination thereof.

In some embodiments, an IL-11R inhibitor as disclosed herein includes, but is not limited to, an anti-IL-11R monoclonal antibody, an oligonucleotide, or a combination thereof.

In some embodiments, the inhibitor is a ROS inhibitor. ROS are chemically reactive molecules containing oxygen, such as, for example, peroxides, superoxide, hydroxyl radical, and singlet oxygen. In some embodiments, the ROS inhibitor is a compound that inhibits mitochondrial ROS generation. In some embodiments, the ROS inhibitor is YCG063 (Structure VII).

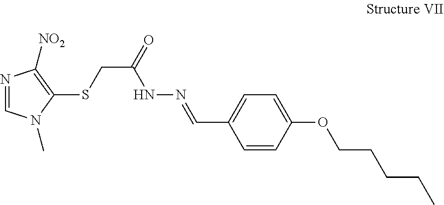 [SMILES: CCCCCOc1ccc(/C=N/NC(=O)CSc2c([N+](=O)[O-])ncn2C)cc1]

In some embodiments, the inhibitor is a ROS scavenger. In some embodiments, the ROS scavenger is a superoxide dismutase and/or catalase mimetic. In some embodiments, the ROS scavenger is manganese(III) tetrakis(1-methyl-4-pyridyl) porphyrin (MnTMPyP).

In some embodiments, a pharmaceutical composition or dosage form as described herein further comprises a pharmaceutically acceptable excipient (e.g., a diluent, carrier, salt or adjuvant). See, e.g., Remington, The Science and Practice of Pharmacy 20th Edition Mack Publishing, 2000. Suitable pharmaceutically acceptable vehicles and/or excipients include, but are not limited to, nontoxic buffers such as phosphate, citrate, and other organic acids; salts such as sodium chloride; antioxidants including ascorbic acid and methionine; preservatives (e.g. octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol); low molecular weight polypeptides (e.g. less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosacchandes, disaccharides, glucose, mannose, or dextrins; chelating agents such as EDTA; sugars such as sucrose, mannitol, trehalose or sorbitol; salt-forming counter-ions such as sodium; metal complexes (e.g. Zn-protein complexes); and non-ionic surfactants such as TWEEN or polyethylene glycol (PEG).

In some embodiments, a pharmaceutical composition or dosing form as disclosed herein further comprises an additional therapeutic agent (e.g., a compound having anti-cancer properties).

Formulations of the pharmaceutical compositions and dosage forms as described herein can be prepared by any method known or developed in the art of pharmacology. In general, such preparatory methods include the step of bringing an active ingredient of the present invention (e.g., a DNA damaging agent, inhibitor, and/or additional therapeutic agent) into association with an excipient and/or one or more other accessory ingredients, and then, if necessary and/or desirable, dividing, shaping and/or packaging the product into a desired single- or multi-dose unit.

Relative amounts of an active ingredient (e.g., a DNA damaging agent, inhibitor, and/or additional therapeutic agent), the pharmaceutically acceptable excipient, and/or any additional ingredients in a pharmaceutical composition or dosage form in accordance with the present disclosure will vary, depending upon the identity, size, and/or condition of the subject treated and further depending upon the route by which the composition is to be administered. By way of example, the composition can comprise between about 0.1% and about 100%, e.g., between about 0.5 and about 50%, between about 1 to about 30%, between about 5 to about 80%, or at least about 80% (w/w) of an active ingredient.

The pharmaceutical compositions and dosage forms of the present invention can be administered in any number of ways for either local or systemic treatment. Administration can be topical (such as to mucous membranes including vaginal and rectal delivery) such as transdermal patches, ointments, lotions, creams, gels, drops, suppositories, sprays, liquids and powders; pulmonary (e.g., by inhalation or insufflation of powders or aerosols, including by nebulizer; intratracheal, intranasal, epidermal and transdermal); oral; or parenteral including intravenous, intraarterial, subcutaneous, intraperitoneal or intramuscular injection or infusion; or intracranial (e.g., intrathecal or intraventricular) administration.

In some embodiments, a pharmaceutical composition or dosage regimen as disclosed herein can provide “synergy” and prove “synergistic”, i.e. the effect achieved when the active ingredients used together is greater than the sum of the effects that results from using the compounds separately. A synergistic effect can be attained when the active ingredients are: (1) co-formulated and administered or delivered simultaneously in a combined pharmaceutical composition or unit dosage form; (2) delivered by alternation or in parallel as separate pharmaceutical compositions or dosage forms; or (3) by some other regimen. When delivered in alternation therapy, a synergistic effect can be attained when the compounds are administered or delivered sequentially, e.g. by different injections in separate syringes. In general, during alternation therapy, an effective dosage of each active ingredient is administered sequentially, i.e. serially, whereas in combination therapy, effective dosages of two or more active ingredients are administered together.

In one aspect, the present invention provides a kit comprising a pharmaceutical composition or dosing regimen as disclosed herein. In some embodiments, the kit comprises a first pharmaceutical composition or dosage form comprising a DNA damaging agent as disclosed herein and a second pharmaceutical composition or dosage form comprising an inhibitor as disclosed herein. In certain embodiments, a kit comprises at least one DNA damaging agent and at least one inhibitor of the invention in one or more containers. In some embodiments, the kit comprises at least one DNA damaging agent and at least one inhibitor in a single pharmaceutical composition or dosage form. In some embodiments, the kit comprises at least one DNA damaging agent and at least one inhibitor as separate pharmaceutical compositions or dosage forms. In some embodiments, the kit comprises a pharmaceutical composition or dosage form comprising one or more DNA damaging agents and a pharmaceutical composition or dosage form comprising one or more inhibitors. In some embodiments, the kit comprises separate pharmaceutical compositions or dosage forms for each individual DNA damaging agent and inhibitor. It will further be appreciated that an additional therapeutic agent can be provided together in a single pharmaceutical composition or dosage form with the DNA damaging agent and/or the inhibitor, or provided separately in different pharmaceutical compositions or dosage forms. In some embodiments, the kit comprises instructions for combined use of the DNA damaging agent and inhibitor. In some embodiments, a kit comprises a DNA damaging agent and an inhibitor as described herein as separate compositions, and the kit further comprises instructions for making a pharmaceutical composition comprising both the DNA damaging agent and inhibitor. In some embodiments, a kit as described herein contains all of the components necessary and/or sufficient for administering the DNA damaging agent, inhibitor, and any additional therapy or therapeutic agent as disclosed herein. One skilled in the art will readily recognize that the disclosed DNA damaging agents and inhibitors of the present invention can be readily incorporated into one of the established kit formats which are well known in the art.

### III. Methods

In one aspect, the present invention is directed to a method of inhibiting the JAK2-STAT5 pathway in a cell, comprising administering to the cell: a) an effective dose of a DNA damaging agent; and b) an effective dose of an inhibitor of the JAK2-STAT5 pathway. In some embodiments, the cell is in vitro. In some embodiments, the cell is in vivo (i.e., in a subject).

In another aspect, the present invention is directed to a method of treating cancer in a subject, comprising administering to the subject: a) an effective dose of a DNA damaging agent; and b) an effective dose of an inhibitor of the JAK2-STAT5 pathway.

In another aspect, the present invention is directed to a method of decreasing resistance to a DNA damaging agent that is used in the treatment of a disease or disorder in a subject, comprising administering to the subject: a) an effective dose of a DNA damaging agent; and b) an effective dose of an inhibitor of the JAK2-STAT5 pathway. In some embodiments, the method is for preventing or delaying development of resistance or tolerance to a DNA damaging agent in a subject. In some embodiments, the method is for reducing or eliminating an existing resistance or tolerance to a DNA damaging agent in a subject. In some embodiments, the method is for treating a disease or disorder in a subject with existing resistance or tolerance to a DNA damaging agent. In some embodiments, the disease or disorder is a cancer.

It is understood that methods of administering a DNA damaging agent and an inhibitor of the JAK2-STAT5 pathway as disclosed herein can alternatively be described as uses of the DNA damaging agent and an inhibitor of the JAK2-STAT5 pathway in the preparation of medicaments, or the DNA damaging agent and an inhibitor of the JAK2-STAT5 pathway for a disclosed use (e.g., for inhibiting the JAK2-STAT5 pathway in a cell, for treating cancer in a subject, or for decreasing resistance to a DNA damaging agent that is used in the treatment of a disease or disorder in a subject).

In the context of treating cancer, an effective dose is, for example, an amount sufficient to reduce or decrease a size of a tumor (i.e., reduce or decrease the size of a tumor mass), decrease the rate of or inhibit a tumor growth, decrease the number of metastases, result in amelioration of one or more symptoms of cancer, prevent advancement of cancer, cause regression of the cancer, increase time to tumor progression, increase tumor cell apoptosis, increase survival time (e.g., increase survival time by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%), or otherwise benefit a subject with cancer as compared to the response obtained without administration of the agent.

In some embodiments, prior to initiation of the method, the subject has been identified as having a cancer that is resistant to treatment with at least one DNA damaging agent. In some embodiments, a method as disclosed herein further comprises determining whether the subject has a cancer that is resistant to treatment with the DNA damaging agent prior to administering the DNA damaging agent and the inhibitor.

In some embodiments, the cancer is selected from the group consisting of: ovarian cancer, testicular cancer, bladder cancer, head and neck cancer, oral cancer, esophageal cancer, lung cancer, small cell lung cancer, non-small cell lung cancer, breast cancer, cervical cancer, stomach cancer, gastric cancer, colorectal cancer, osteosarcoma, pancreatic cancer, prostate cancer, and any combination thereof. In some embodiments, the cancer is ovarian cancer.

In some embodiments, the DNA damaging agent can be administered prior to, concurrently with, or subsequent to the inhibitor.

The DNA damaging agent and inhibitor of the methods can be any DNA damaging agent and inhibitor as described above with respect to the pharmaceutical compositions and dosing regimens of the invention.

In some embodiments, a method as disclosed herein comprises an inhibitor selected from the group consisting of: a JAK2 inhibitor, a STAT5 inhibitor, an interleukin-11 (IL-11) inhibitor, a Fos-related antigen 1 (FRA1) inhibitor, a reactive oxygen species (ROS) inhibitor, a ROS scavenger, and any combination thereof.

In some embodiments, a method as disclosed herein comprises a JAK2 inhibitor selected from the group consisting of: LY2784544, TG101348, TG46, and any combination thereof.

In some embodiments, a method as disclosed herein comprises an IL-11 inhibitor selected from the group consisting of: an anti-IL-11 monoclonal antibody, an anti-IL-11 receptor monoclonal antibody, and a combination thereof.

In some embodiments, a method as disclosed herein comprises a ROS inhibitor, a ROS scavenger, or a combination thereof, wherein the ROS inhibitor is YCG063 and the ROS scavenger is MnTMPyp.

In some embodiments, a method as disclosed herein comprises administering a pharmaceutical composition, a dosing regimen, or a dosage form as described herein.

In some embodiments, the DNA damaging agent is a platinum-based drug. In some embodiments, the platinum-based drug is selected from the group consisting of: cisplatin, carboplatin, diplatinum cytostatic, iproplatin, oxaliplatin, nedaplatin, satraplatin, tetraplatin, and any combination thereof.

In some embodiments, prior to initiation of the method, the level of IL-11 mRNA or IL-11 protein, reactive oxygen species (ROS), or any combination thereof in cells or blood serum in the subject is higher than in control cells or blood serum. In some embodiments, a method as disclosed herein further comprises determining the level of IL-11 mRNA or IL-11 protein, ROS, or any combination thereof in cells or blood serum of the subject prior to administering the DNA damaging agent and the inhibitor. In some embodiments, a method as disclosed herein further comprises determining the level of IL-11 mRNA or IL-11 protein in cells or blood serum of the subject prior to administering the DNA damaging agent and the inhibitor. In some embodiments, a method as disclosed herein further comprises determining the level of ROS in cells or blood serum of the subject prior to administering the DNA damaging agent and the inhibitor. As used herein, the term “determining the level of ROS” can be used interchangeably with the term “determining the level of oxidative stress.” In some embodiments, the cells of the subject are cancer cells. The control cells or blood serum can be any standard or acceptable control with respect to the disease or disorder being treated (e.g., a cancer including, but not limited to, ovarian cancer).

In some embodiments, a method as disclosed herein comprises administering to the subject an effective dose of LY2784544 (Gandotinib) and an effective dose of cisplatin, wherein the combination of LY2784544 and cisplatin result in a synergistic effect as compared to treatment with either drug alone.

In some embodiments, a method as disclosed herein further comprises administering one or more other additional therapies or therapeutic agents.

The DNA damaging agent, inhibitor, and any other additional therapeutic agent in a method as disclosed herein can be administered in any order. In general, each agent (i.e., each DNA damaging agent, inhibitor, and any other additional therapeutic agent) will be administered at a dose and/or on a time schedule determined for that agent. It will further be appreciated that an additional therapeutic agent can be administered together in a single pharmaceutical composition or dosage form with the DNA damaging agent and/or inhibitor, or administered separately in a different pharmaceutical composition or dosage form. In general, it is expected that an agent will be utilized at a level in the methods that does not exceed the level at which the agent is utilized individually. In some embodiments, the level of agent utilized in the methods will be lower than the level of the agent utilized individually.

The DNA damaging agent, inhibitor, and/or any additional therapeutic agent in a method as disclosed herein can be manufactured and/or formulated by the same or different manufacturers. The DNA damaging agent, inhibitor, and/or any additional therapeutic agent can thus be entirely separate pharmaceutical compositions or dosage forms. In some embodiments, instructions for their combined use are provided: (i) prior to release to physicians (e.g., in a “kit” comprising the DNA damaging agent, inhibitor, and any additional therapeutic agent); (ii) by the physicians themselves (or under the guidance of a physician) shortly before administration; or (iii) to the patient themselves by a physician or medical staff.

### EXAMPLES

Reference is now made to the following examples, which together with the above descriptions illustrate some embodiments of the invention in a non-limiting fashion.

### Example 1

### Generation of Cisplatin Resistant Ovarian Cancer Cells

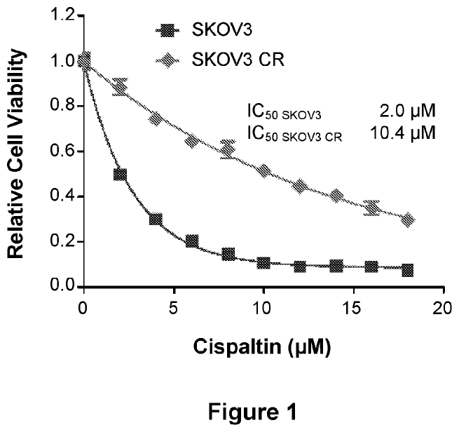

Cisplatin resistant ovarian cancer cell lines were generated using procedures as described previously. Jazaeri et al., Mol Cancer Ther 12: 1958-1967 (2013). Ovarian cancer cells SKOV3 were cultured in the medium with cisplatin (Sigma-Aldrich) for three weeks, followed by release to cisplatin-free medium for another three weeks. In the next cycle, the cisplatin treatment was repeated in the medium with an increased concentration of cisplatin. After six-cycle treatment, cells were identified that were able to grow in the medium with a high concentration of cisplatin. These cisplatin resistant cells were named as SKOV3 CR (FIG. 1). A cell viability assay revealed that the IC₅₀ of SKOV3 CR was increased to 10.4 μM from 2.0 μM of parental cells SKOV3 (FIG. 1).

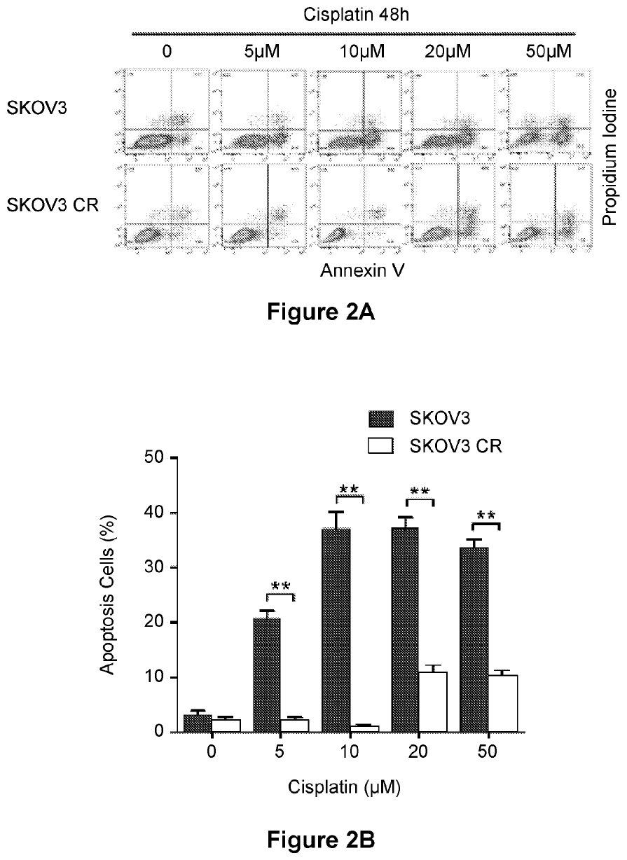

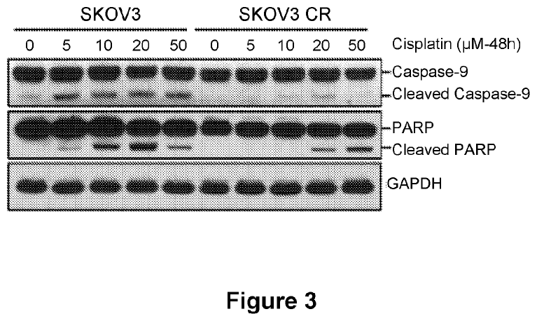

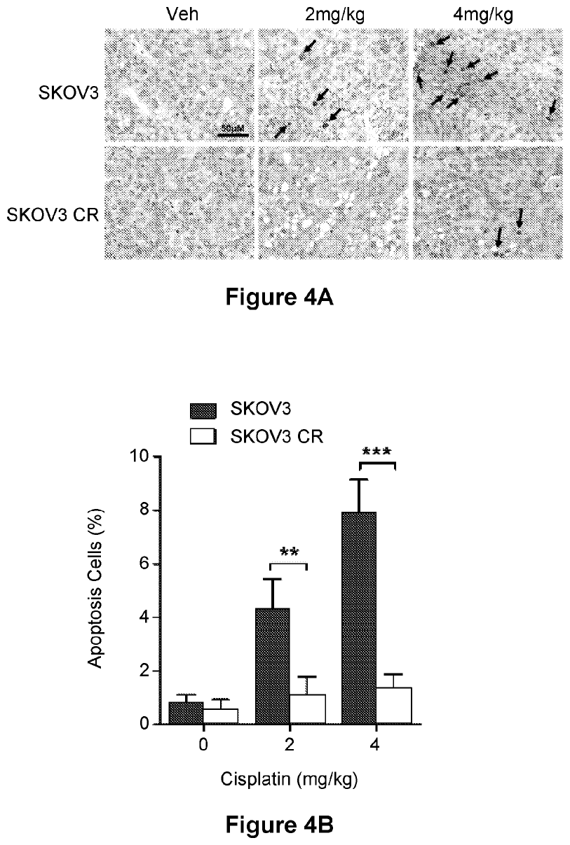

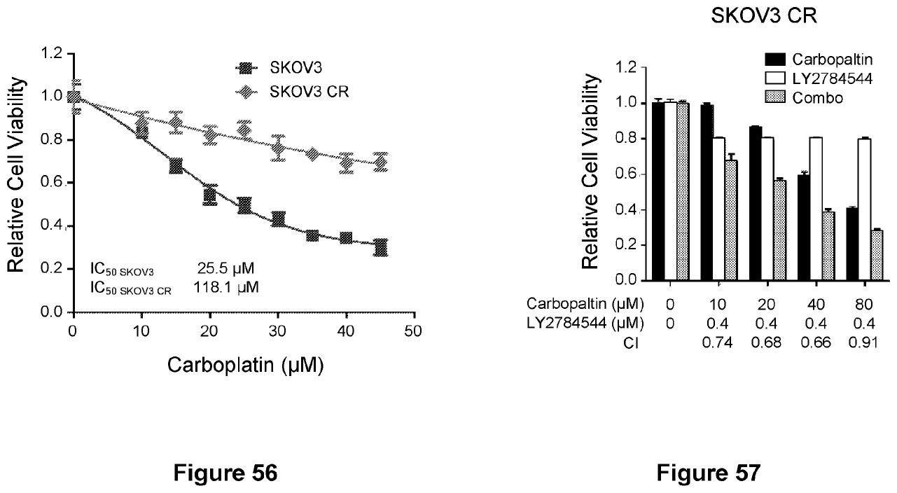

Since cisplatin exerts its anticancer effect mainly through its ability to cause DNA damage, resulting in apoptosis, the apoptotic cells in both SKOV3 and SKOV CR cells were measured using Annexin V and Propidium Iodide staining. The results showed that cisplatin induced more apoptosis in SKOV3 cells than in SKOV3 CR cells (FIG. 2). Consistently, a significant increase of cleavages of poly (ADP-ribose) polymerase (PARP) and Caspase-9 in SKOV3 were detected as compared to SKOV3 CR cells (FIG. 3), indicating that SKOV3 CR cells are more resistant to apoptotic cell death in response to cisplatin. To test whether SKOV3 CR cells are resistant to cisplatin-induced apoptosis in vivo, both SKOV3 and SKOV3 CR cells were injected into nude mice to form xenograft tumors. Once the tumor size reached 100-150 mm³, cisplatin was given to mice by intraperitoneal injections twice a week for 2 weeks. Three days after treatment, the apoptosis in tumors was examined by TUNEL staining. It was found that the apoptotic population was significantly increased in SKOV3 but not in SKOV3 CR cells (FIG. 4). Currently, carboplatin is a more commonly used platinum drug employed clinically to treat ovarian patients. Ozols et al., J Clin Oncol 21: 3194-3200 (2003). Thus, it was tested whether SKOV3 CR cells are also resistant to carboplatin (Sigma-Aldrich). The IC₅₀ of SKOV3 CR (118.1 μM) was increased 4-fold compared to parental cells SKOV3 (25.2 μM) as shown in FIG. 56.

These results demonstrate the generation of cisplatin resistant ovarian cells SKOV3 CR that are resistant to platinum drugs both in vitro and in vivo.

### Example 2

### Identification of JAK2 Inhibitor LY2784544 that Overcomes Cisplatin Resistance via a Combinational High Throughput Screen

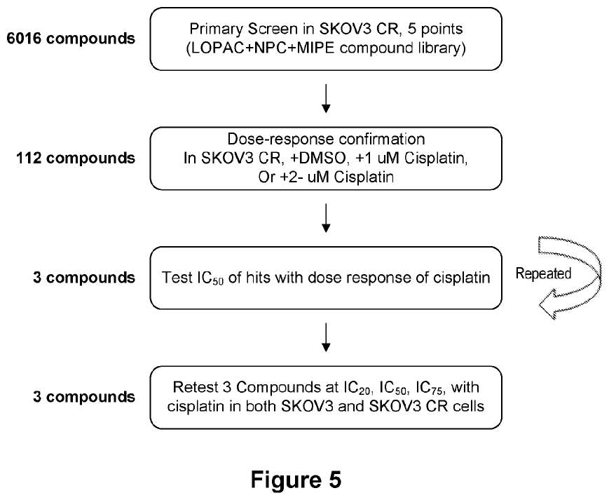

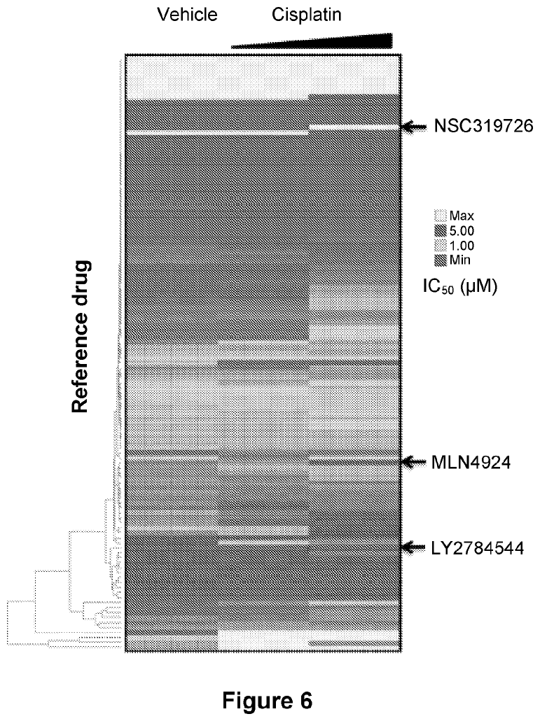

A combinational HTS using multiple compound libraries including NPC (NIH Pharmaceutical Collection), MIPE (Mechanism interrogation plate), and LOPAC (The Library of Pharmacologically Active Compounds) was used to identify compounds that can overcome cisplatin resistance of SKOV3 CR cells (FIG. 5). The library of pharmacologically active compounds (LOPAC®) of 1280 compounds (1) was purchased from Sigma. The NCGC Pharmaceutical Collection (NPC) of 2816 compounds and mechanism interrogation plate (MIPE) library of 1920 compounds were previously described. Bromberg, J Clin Invest 109: 1139-1142 (2002) and Buchert et al., Oncogene 35: 939-951 (2016). Compounds from all libraries were obtained as powder and dissolved in dimethyl sulfoxide (DMSO) using Prism software (GraphPad). Combinational HTS was performed as previously described. Szasz et al., Oncotarget 7: 49322-49333 (2016). A total of 6,016 compounds were screened. At the first screen, SKOV3 CR cells grown in 1536-well plates were treated with compounds at five different concentrations, and a total of 112 compounds were identified that efficiently inhibit the proliferation of SKOV3 CR. In the second screen, these 112 compounds were selected for a dose-response confirmation screen in combination with DMSO, 1 μM cisplatin or 20 μM cisplatin (FIG. 5). From this second screen, three compounds LY2784544 (JAK2 inhibitor), MLN4924 (Neddylation inhibitor) and NSC319726 (p53 mutant reactivator) were found to significantly sensitize SKOV3 CR cells to cisplatin (FIG. 6).

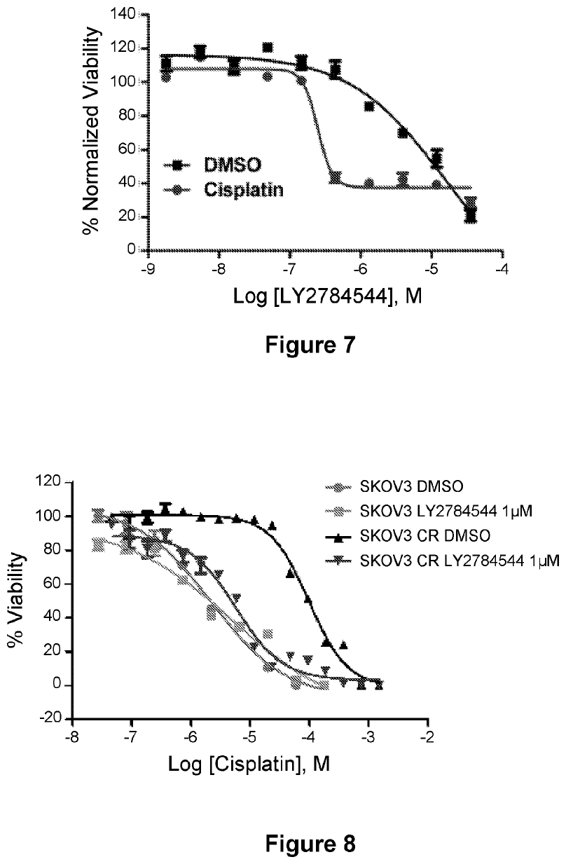

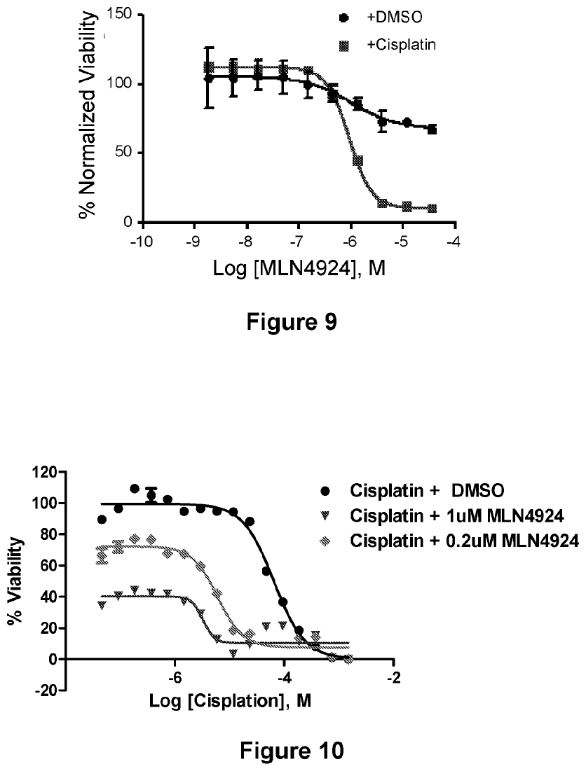

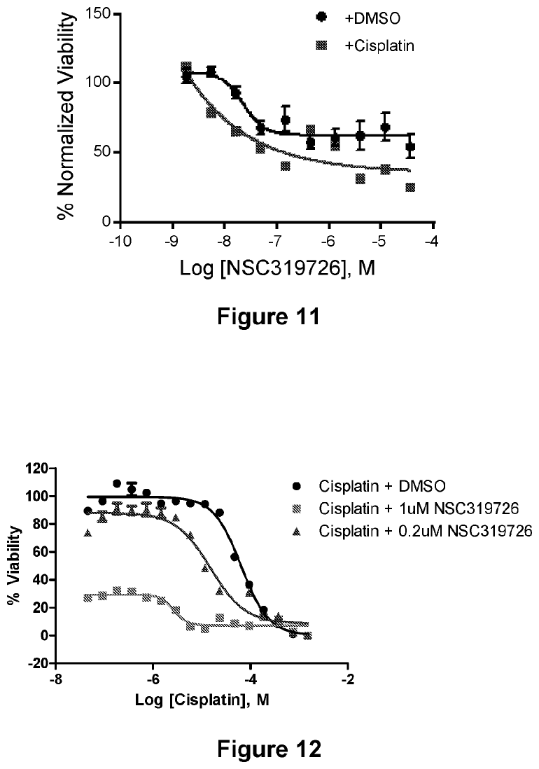

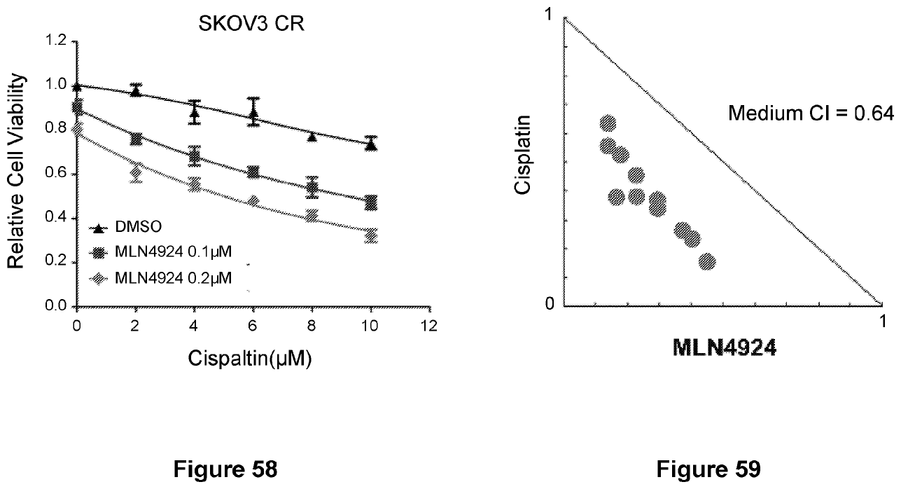

The effects of these compounds on the proliferation of both SKOV3 and SKOV3 CR in combination with cisplatin at different doses were tested. Although it did not affect the cell proliferation of SKOV3 in the medium with cisplatin or without cisplatin, LY2784544 (Selleckchem) significantly increased the sensitivity of SKOV3 CR cells to cisplatin to the degree similar to SKOV3 cells. Interestingly, cisplatin could sensitize SKOV3 CR cells to LY2784544 reciprocally (FIGS. 7 and 8), indicating the potential synergistic effects of LY2784544 and cisplatin. Similar combinational studies were performed using MLN4924 and NSC319746. Significantly, both MLN4924 and NSC319746 can sensitize SKOV3 CR cells to cisplatin and interestingly cisplatin also sensitizes SKOV3 CR cells to MLN4924 or NSC319746, indicating the synergistic effects of both MLN4924 and NSC319746 with cisplatin (FIGS. 9-12). Using cell proliferation assays, the synergistic effects of MLN4924 with cisplatin on SKOV3 CR cells (FIGS. 58 and 59) was confirmed. The synergistic effect of both MLN4924 or NSC319746 on cisplatin resistant ovarian cancer cells have been reported independently by other groups. Jazaeri et al., Mol Cancer Ther 12: 1958-1967 (2013) and Yu et al., Cancer Cell 21: 614-625 (2012). Among the three compounds, LY2784544 has better cisplatin IC50 shift ability than MLN4924 or NSC319726, which significantly increased the sensitivity of SKOV3 CR cells to cisplatin to a degree similar to SKOV3 cells.

### Example 3

### The JAK2 Inhibitor LY2784544 Synergizes with Platinum Drugs in Ovarian Cancer Cisplatin Resistant Cells In Vitro

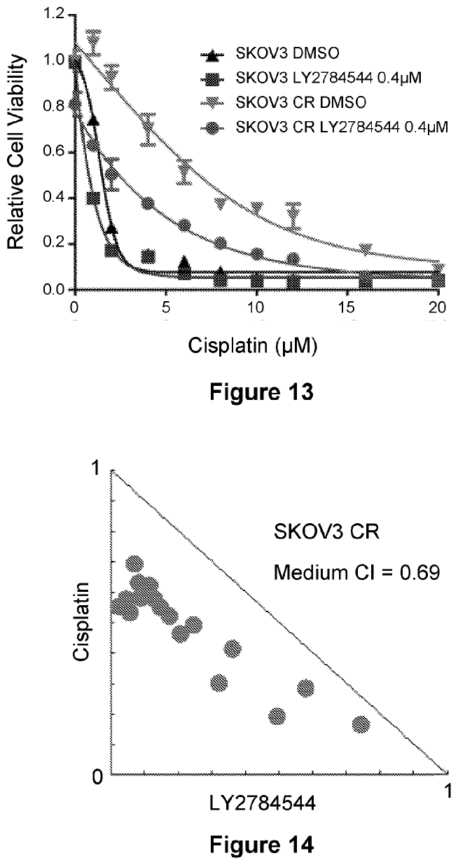

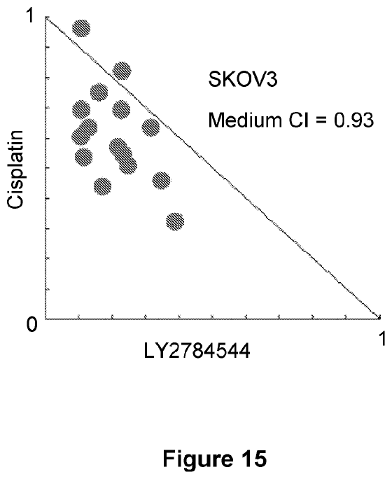

The results obtained from combinational HTS were confirmed by testing whether LY2784544 overcomes cisplatin resistance in ovarian cancer cells using multiple assays. By measuring cell proliferation using the sulforhodamine B (SRB) assay (Wu et al., Cancer Cell 28: 29-41 (2015)), it was found that LY2784544 re-sensitized SKOV3 CR cells to cisplatin (FIG. 13), consistent with data obtained from HTS. The Combination index (CI), which quantitatively describes the interaction between drugs including synergism (CI<1), additive effect (CI=1), and antagonism (CI>1), was calculated. The combination of cisplatin and LY2784544 exhibited excellent synergy on both SKOV3 CR cells (medium CI=0.69) as well as SKOV3 cells (medium CI=0.93) (FIGS. 14 and 15), indicating that LY2784544 has synergistic effects with cisplatin on both SKOV3 CR and SKOV3 cells.

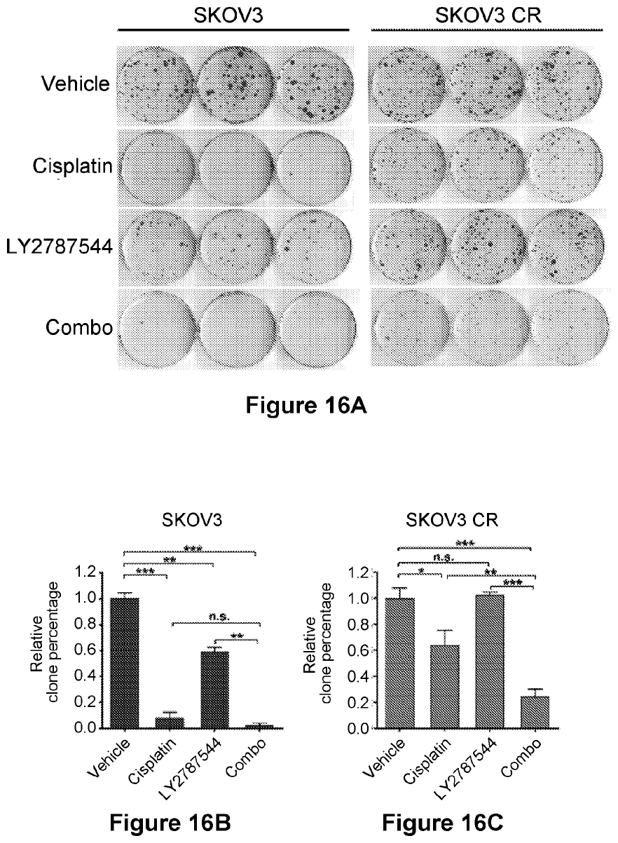

Clonogenic assays were conducted to evaluate the cell survival of SKOV3 and SKOV3 CR cells in response to cisplatin, LY2784544, or both cisplatin and LY2784544. After two weeks of treatment, SKOV3 CR cells exhibited a higher survival rate compared to SKOV3 cells, and combined treatment of cisplatin and LY2784544 significantly reduced the survival of SKOV3 CR compared to cisplatin treatment (FIG. 16). Although cisplatin-based chemotherapy in ovarian cancer had a clear advantage in ovarian cancer patients OS (overall survival) and PFS (progression free survival), the peripheral neurotoxicity and renal toxicity limit its use. Currently, carboplatin-based chemotherapy becomes a universal choice. The synergistic effect of LY2784544 with carboplatin on SKOV3 CR cells was also examined. As shown in (FIG. 57), the combination of carboplatin and LY2784544 exhibited synergy (medium CI=0.75) on SKOV3 CR cells, indicating that the synergistic effect of LY2784544 with platinum drugs are not limited to cisplatin.

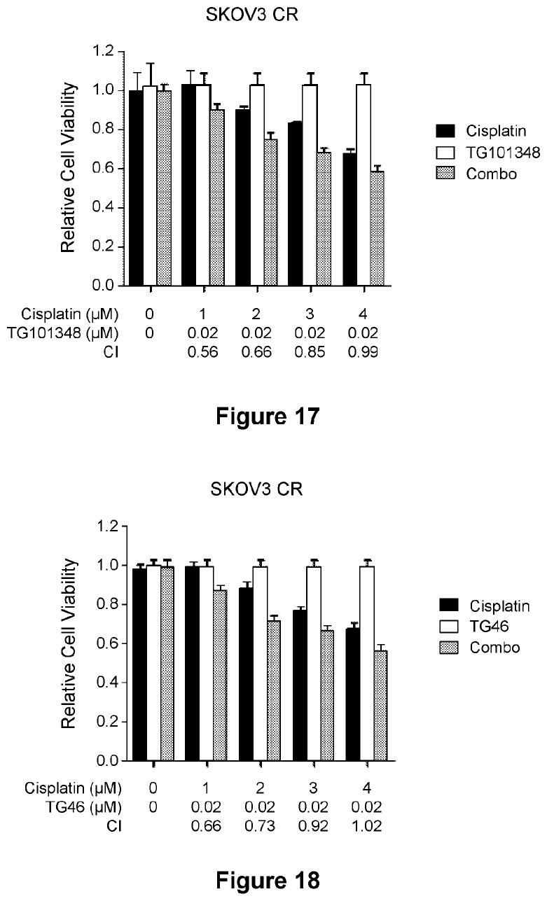

Considering the off-target effects of compounds, two other JAK2 inhibitors—TG101348 and TG46, purchased from Selleckchem and SynKinase, respectively—were tested for synergistic effects with cisplatin on SKOV3 CR cells. Like LY2784544, TG101348 and TG46 are selective JAK2 inhibitors vs JAK1 and JAK3. Both TG101348 and TG46 showed a synergistic effect (TG101348 medium CI=0.77, TG46 medium CI=0.77) with cisplatin on SKOV3 CR cells (FIGS. 17 and 18).

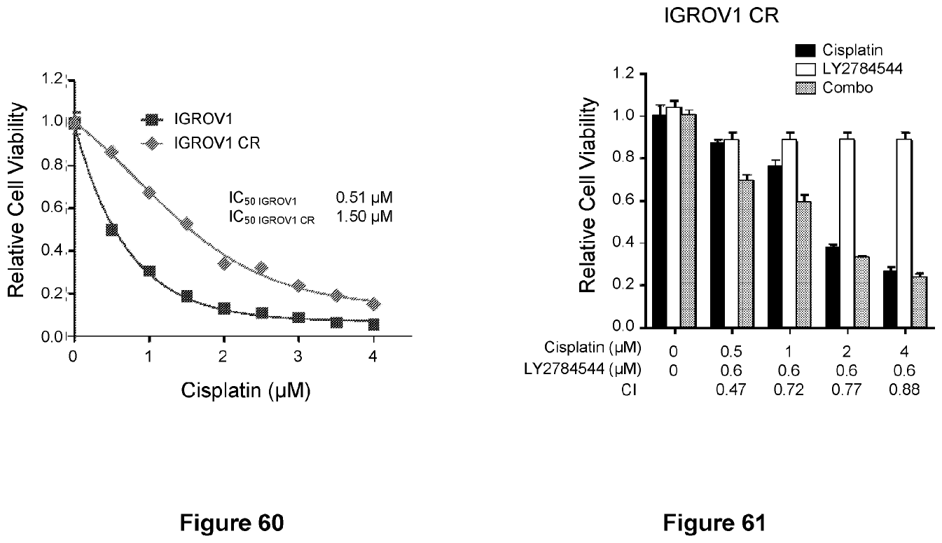

Using a similar approach as described in FIG. 1, another cisplatin resistant ovarian cancer cell line IGROV1 CR was raised from its cisplatin sensitive parental cell IGROV1 cells. The constant cisplatin treatment increased the IC₅₀ by 2.9-fold from 0.51 μM in parental IGROV1 cells to 1.50 μM in cisplatin resistant IGROV1 CR cells (FIG. 60). The cell proliferation of IGROV1 CR cells treated with cisplatin, LY2784544 or both LY2784544 and cisplatin was examined. It was found that combination of cisplatin and LY2784544 exhibited excellent synergy (medium CI=0.71) on IGROV1 CR cells, indicating that the synergistic effect of a combination of cisplatin and LY2784544 on cisplatin resistant ovarian cancer cells is not limited to a single resistant cell line (FIG. 61).

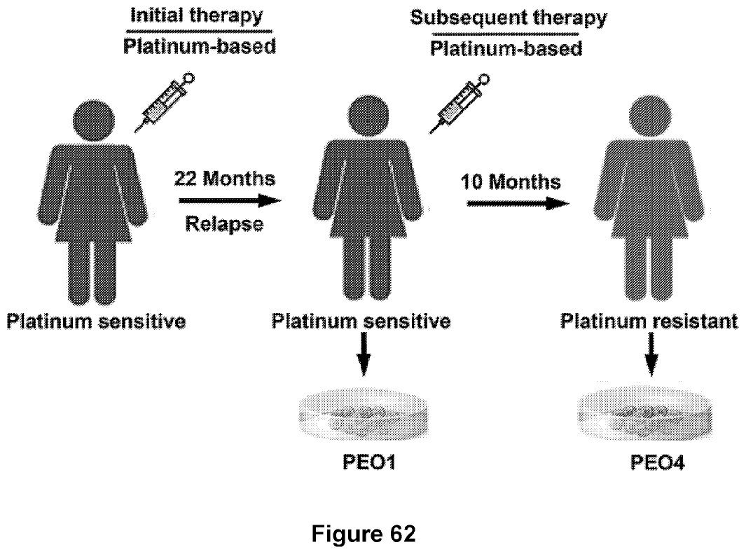

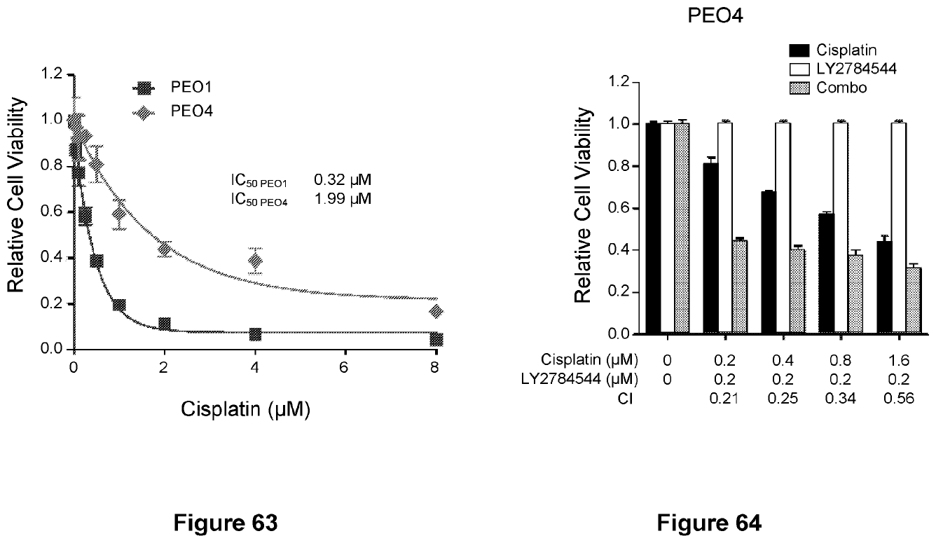

A paired ovarian cancer cell line derived from the same patient before and after chemotherapy was used to show that LY2784544 overcomes cisplatin resistance. As shown in FIG. 62, PEO1 are collected from a patient with tumor relapse 22 months after cisplatin based chemotherapy. For ovarian cancer, recurrence more than 12 months after initial chemotherapy was considered as platinum sensitive. PEO4 was derived from the patient 10 months later when they had progressive disease and had become cisplatin resistant. The IC₅₀ was increased by 6.2-fold from 0.32 μM in PEO1 cells to 1.99 μM in PEO4 cells (FIG. 63). The combination of cisplatin and LY2784544 also exhibited synergy (medium CI=0.34) on PEO4 CR cells, indicating that the synergistic effect of a combination of cisplatin and LY2784544 on cisplatin resistant ovarian cancer cells is consistent in patient derived platinum resistant cells (FIG. 64).

### Example 4

### LY2784544 Overcomes Cisplatin Resistance of Ovarian Cancer Cells by Inhibiting JAK2-Mediated Pathway In Vitro and In Vivo

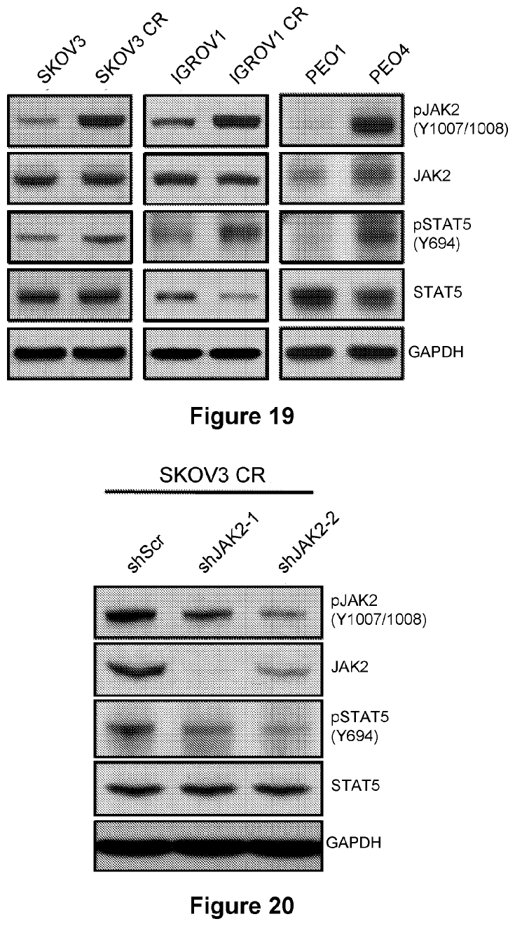

To investigate the molecular mechanism by which the JAK2-mediated pathway regulates cisplatin resistance of ovarian cancer, the activation of the JAK2-mediated pathway in paired sensitive cell line (SKOV3, IGROV1, PEO1) and resistant cell lines (SKOV3 CR, IGROV1 CR, PEO4) was examined by western blot. Compared to cisplatin sensitive cells SKOV3 or IGROV1, the JAK2 protein levels are not changed in cisplatin resistant cells SKOV3 CR, IGROV1 CR, or PEO4. The phosphorylation of JAK2 at Y1007/1008 was significantly increased in all resistant cells SKOV3 CR, IGROV1 CR, and PEO4 compared to their sensitive counterparts, indicating the JAK2 kinase is activated in cisplatin resistant ovarian cells (FIG. 19). Consistent to the activation of JAK2, it was found that the phosphorylation of STAT5 (Y694), a downstream target of JAK2, was increased in all three cell lines (i.e., SKOV3 CR, IGROV1 CR, and PE04), indicating that the JAK2 pathway is constitutively activated in cisplatin resistant ovarian cells.

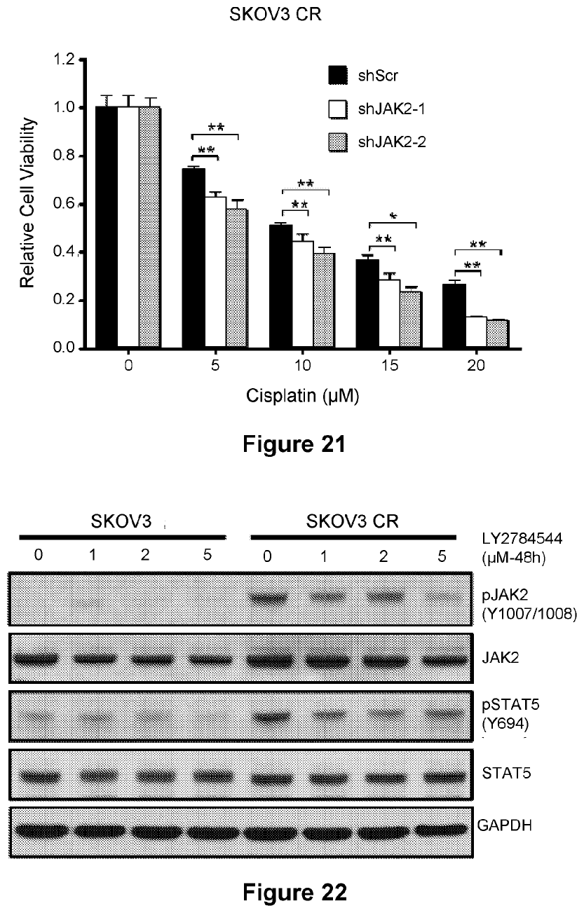

To confirm that cisplatin resistance observed in cisplatin resistant ovarian cells is indeed due to the activation of JAK2-mediated pathway, JAK2 was silenced by two independent shRNAs (shJAK2-1 and shJAK2-2). The downregulation of JAK2 significantly reduced the JAK2 protein levels as well as phosphorylation of STAT5 (FIG. 20). Consistent with data obtained by JAK2 inhibitors, downregulation of JAK2 by two shRNAs also sensitized the SKOV3 CR cells to cisplatin, indicating that the cisplatin resistance in SKOV3 CR is JAK2-dependent (FIG. 21).

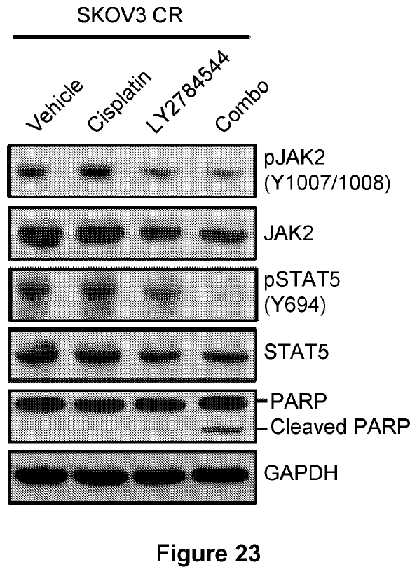

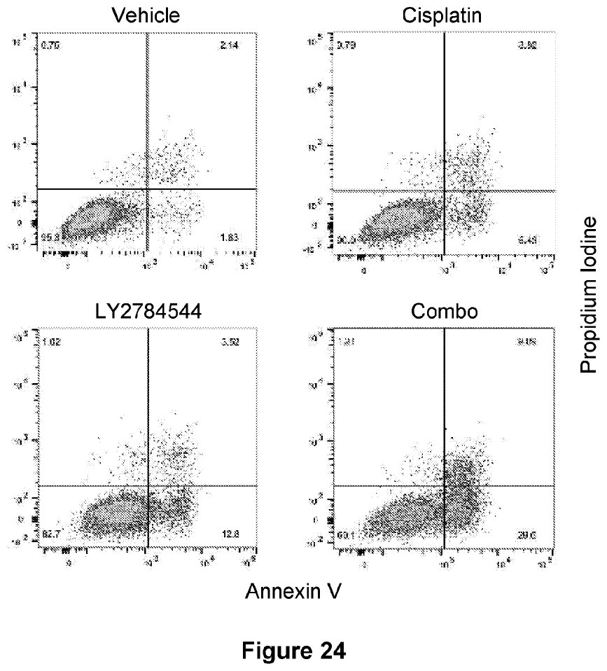

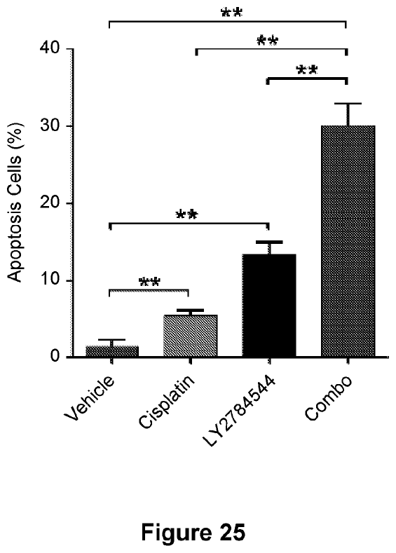

The effects of LY2784544 on the JAK2/STAT5 signaling pathway in both SKOV3 and SKOV3 CR cells were examined. LY2784544 significantly reduced the phosphorylation of JAK2 as well as the phosphorylation of STAT5 in both SKOV3 and SKOV3 CR cells (FIG. 22), indicating that LY2784544 is an inhibitor of JAK2. Since combination of LY2784544 and cisplatin significantly inhibits the cell proliferation, the JAK2/STAT5 pathway in SKOV3 CR cells treated with cisplatin, LY2784544 or both was examined. Consistent with results shown in FIG. 22, LY2784544 efficiently decreased the activation of JAK2 and STAT5 in SKOV3 CR cells but did not cause apoptosis, as indicated by a lack of cleaved poly (ADP-ribose) polymerase (PARP). However, the combination of cisplatin and LY2784544 not only reduced the phosphorylation of JAK2 and STAT5, but also induced apoptotic cell death as indicated by generation of cleaved PARP (FIG. 23). The apoptosis induced by combination of cisplatin and LY2784544 in SKOV3 CR cells was also confirmed by Annexin V and Propidium Iodide staining, which shows a significant increase of apoptotic cells (FIGS. 24 and 25).

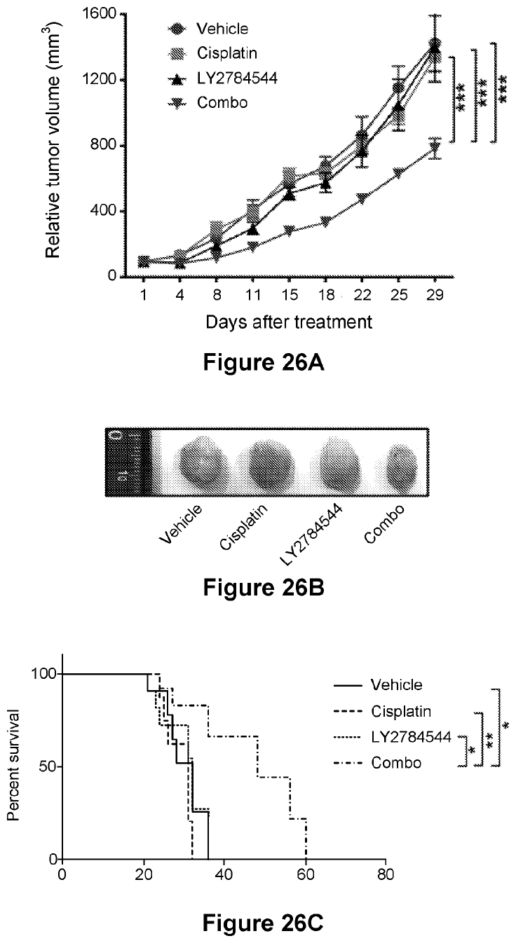

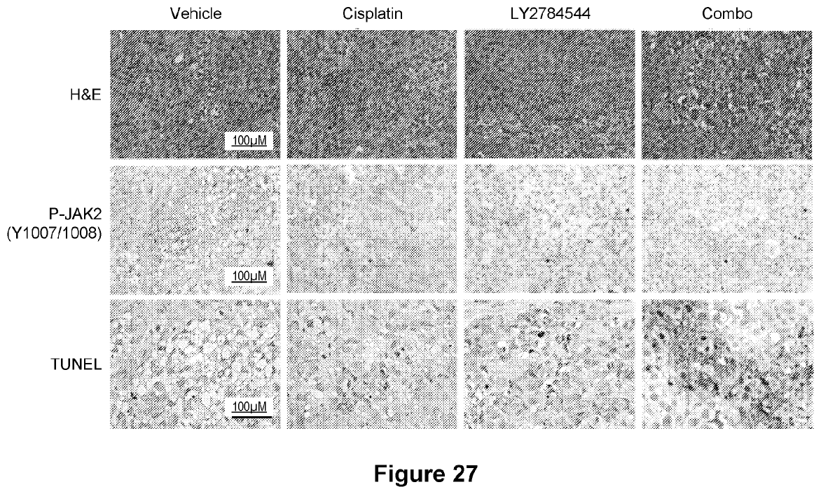

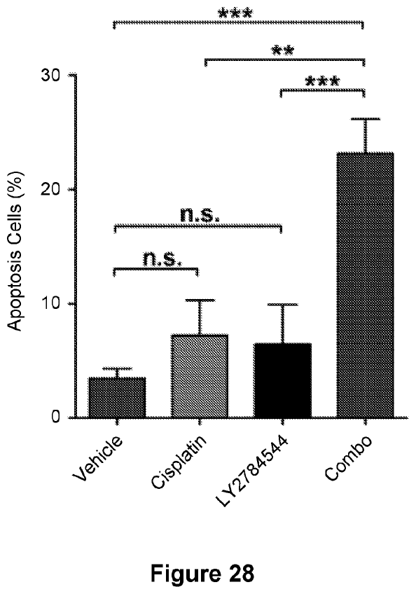

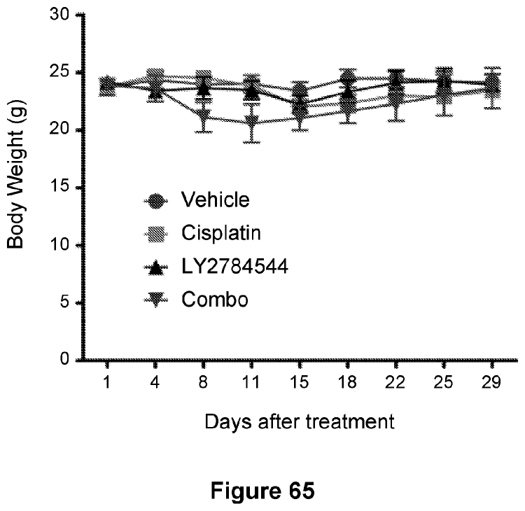

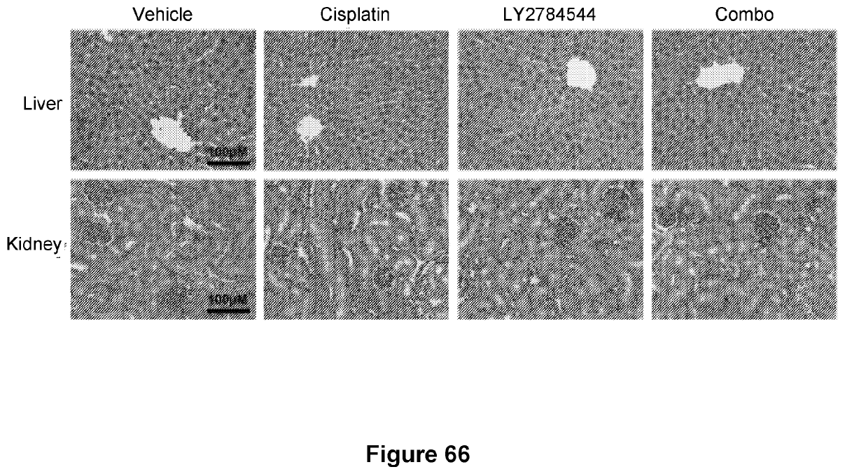

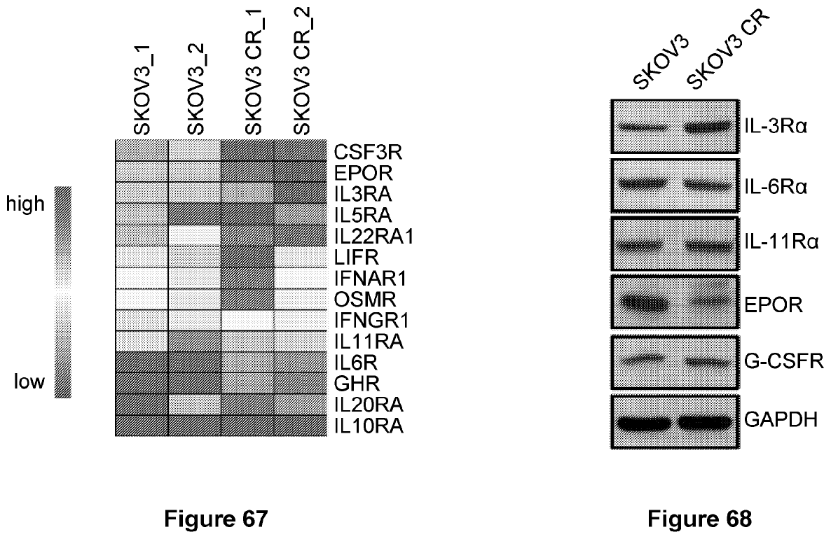

To determine whether LY2784544 is able to sensitize SKOV3 CR cells to cisplatin in vivo, SKOV3 CR cells were implanted subcutaneously into nude mice. When the tumor volume reached 100 mm³, mice were randomized to receive intraperitoneal injections of vehicle, cisplatin (8 mg/kg on days 1 and 8), LY2784544 (15 mg/kg/d from day 1 to day 14), or the combination of cisplatin and LY2784544. Compared to vehicle, or cisplatin or LY2784544 alone, combined cisplatin and LY2784544 significantly reduced the tumor growth (FIG. 26A) and also improved survival of the mice with SKOV3 CR tumors (FIG. 26C). To evaluate whether this in vivo tumor suppression was through inhibition of the JAK2 pathway and increased apoptosis, the tumor samples four days after full dosage treatment were collected. IHC analyses of SKOV3 CR xenograft tumors indicated that LY2784544 can efficiently reduce the phosphorylation of JAK2 in vivo and combined LY2784544 with cisplatin significantly induced the apoptosis as indicated by TUNEL staining (FIG. 27). Quantification of phosphorylated JAK2 and TUNEL staining in tumor tissues treated was performed (FIG. 28). To evaluate the toxicity of combined treatment of cisplatin and LY2784544 on mice, weight of mice during these treatments was monitored and it was found that there was no significant reduction in mouse body weight (FIG. 65). No histopathological changes and lesions in liver and kidney in any treated groups were found (FIG. 66). To confirm the selectivity of LY2784544, an in vitro kinase profiling was conducted to examine the interaction of LY2784544 with more than 450 human kinases. The results showed that LY2784544 exhibited stronger binding to JAK2 than JAK1 and TYK2 (data not shown), indicating the high selectivity of LY2784544 towards JAK2.

### Example 5

### IL-11 is the Major Autocrine Factor for JAK2/STAT5 Activation and Cisplatin Resistance in Ovarian Cancer

The detailed molecular mechanism governing the activation of JAK2 in cisplatin resistant ovarian cancer cells was investigated. Given that JAK2 could be constitutively activated by mutation at V617F or by other mutations, the mutation of JAK2 gene by sequencing the JAK2 exon of both SKOV3 and SKOV3 CR cells was examined, and no mutations in SKOV3 CR cells were found.

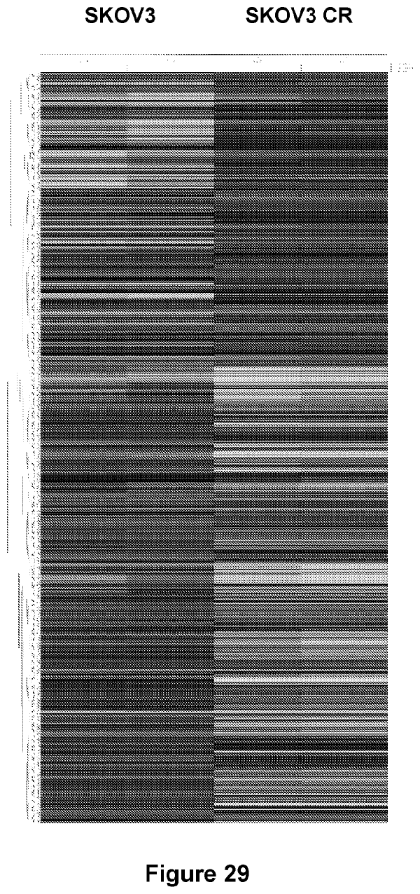

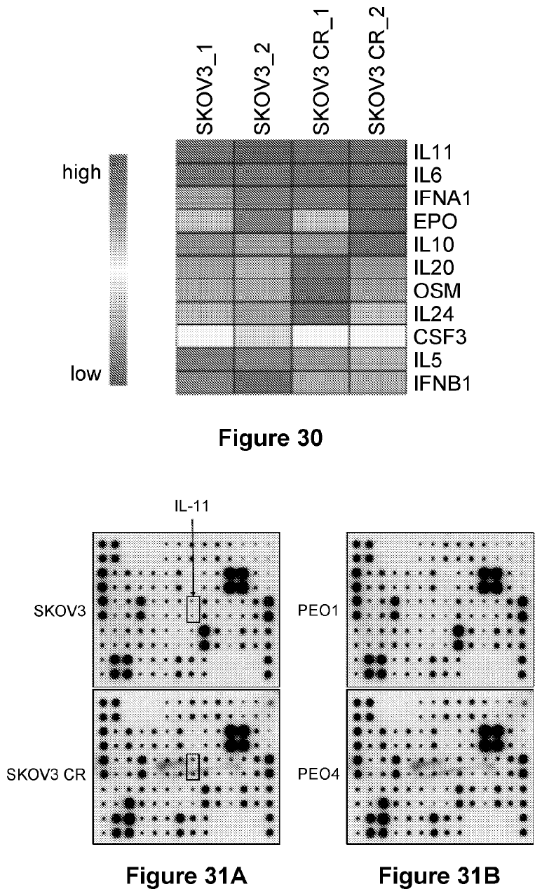

To identify genes or pathways that regulate the activation of JAK2 in cisplatin resistant SKOV3 CR cells, gene expression profiles from both SKOV3 and SKOV3 CR cells were examined and compared. A total of 1086 genes are up-regulated and a total of 1686 genes are down-regulated in SKOV3 CR compared to SKOV3 cells (FIG. 29). Given that the cytokine pathway regulates the activation of JAK2, the gene expression of JAK-related cytokines was determined. It was found that a total of 11 genes are up- or down-regulated in SKOV3 CR cells (FIG. 30). To identify specific cytokine genes that can regulate the activation of JAK2 in the ovarian cancer resistant cells, a cytokine array in SKOV3 and SKOV3 CR cells as well as PEO1 and PEO4 cells was performed. Cytokine arrays on cell culture medium were performed using the Human Cytokine Antibody Array Kit (Abeam, 120 Targets) according to the manufacture's protocol. Single intensity was analyzed using ImageJ (NIH) software. The results were then normalized using internal controls, and the relative protein levels were determined across four biological replicates. The results show that of all up-regulated cytokines, IL-11 is the only up-regulated cytokine in both resistant cell lines (FIG. 31).

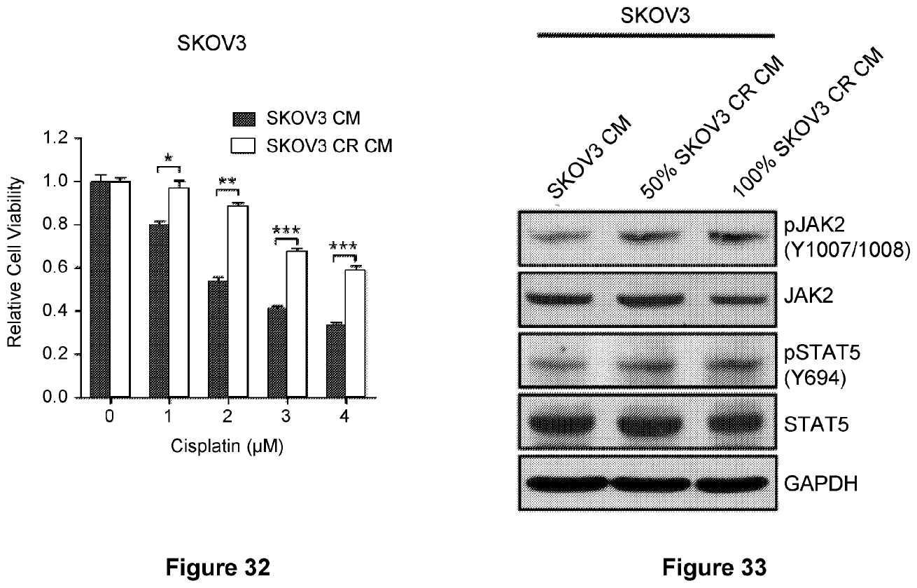

If the cytokine pathway regulates cisplatin resistance of ovarian cancer cells, secreted factors in the medium from SKOV3 CR cells may cause the resistance of SKOV3 cells to cisplatin. To test this possibility, conditional medium was collected from SKOV3 CR cells, which then was applied to grow SKOV3 cells. The conditional medium from SKOV3 cells served as a control. SKOV3 cells supplemented with conditional medium from SKOV3 CR cells displayed an increased resistance to cisplatin compared to SKOV3 cells supplemented with conditional medium from SKOV3, indicating that secreted factors from SKOV3 CR cells cause the cisplatin resistance of SKOV3 cells (FIG. 32). Western blot results indicated that the conditional medium from SKOV3 CR cells can activate JAK2/STAT5 pathway in SKOV3 cells (FIG. 33).

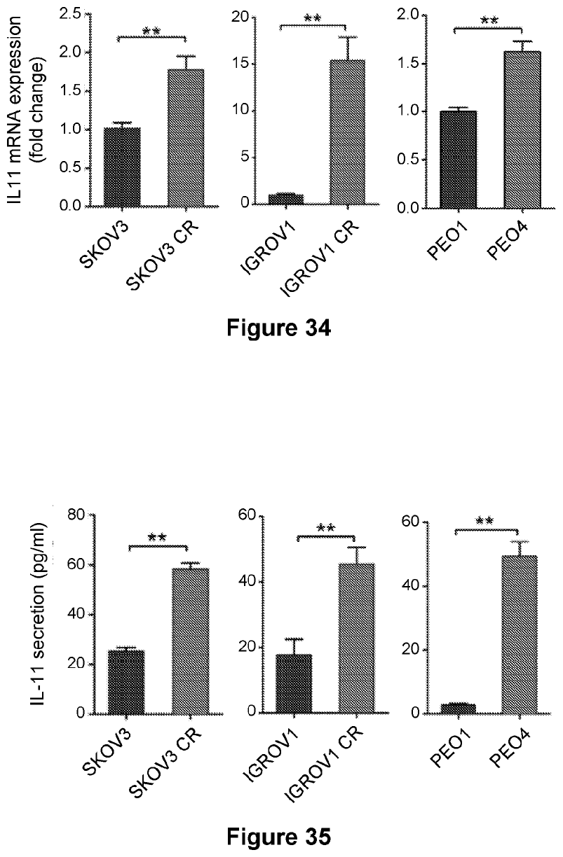

Next, the factor from medium that contributes to cisplatin resistance of SKOV3 CR cells was investigated. IL-11 was found to be up-regulated by RNA-seq analysis, and IL-11 mRNA levels were measured by qPCR. mRNA levels of IL-11 were significantly increased in cisplatin resistant cells SKOV3 CR, IGROV1 CR and PEO4 compared to their sensitive counterparts (FIG. 34). Consistently, using ELISA, it was found that secreted IL-11 in the medium of cisplatin resistant cells SKOV3 CR, IGROV1 CR and PEO4 was increased compared to their sensitive counterparts (FIG. 35). The cell-free culture medium, mouse serum, and patient serum were analyzed for IL-11 levels using a human IL-11 ELISA kit (R&D Systems) according to the manufacturer's instructions. The absorbance was read at 450 nm using a microplate reader (BioTek). The data were normalized to the cell number. To rule out the possibility that JAK2-STAT5 activation in the resistant cells might be due to the overexpression of IL-11 receptors, the expression of subunits of the IL-11 receptor, including IL-11Rα, IL-6Rα, EPOR and G-CSFR, were examined in SKOV3 and SKOV3 CR cells. It was found that the levels of each protein did not increase in SKOV3 CR compared to SKOV3 cells (data not shown).

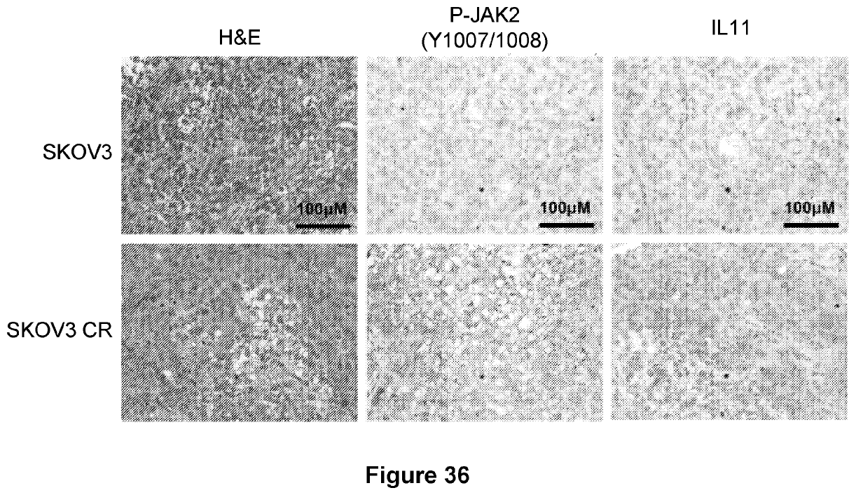

Given that IL-11 mRNA is increased in resistant cells and IL-11 protein is elevated in the medium of resistant cells, the expression of IL-11 in cisplatin resistant ovarian tumors in vivo was investigated. The expression of IL-11 was examined by HIS, and it was found that IL-11 as well as phosphorylation of JAK2 levels were increased in SKOV3 CR tumor compared to that in SKOV3 tumor (FIG. 36). These results indicate that IL-11 levels are up-regulated in cisplatin resistant cancer cells both in vitro and in vivo.

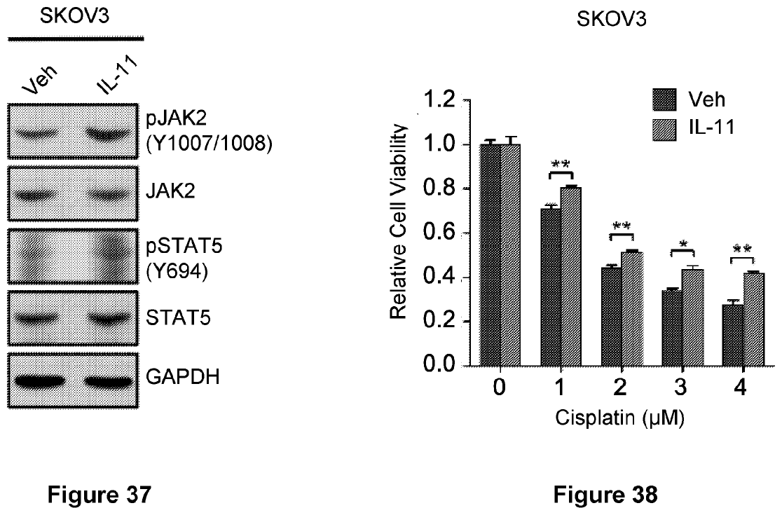

To investigate the molecular functional link between the IL-11 and JAK2 pathways, it was determined whether IL-11 can stimulate the activation of JAK2. To this end, recombinant IL-11 was added to the medium of cisplatin sensitive cells SKOV3. The addition of IL-11 did increase the phosphorylation of JAK2 (Y1007/1008) as well as phosphorylation of STAT5 (Y694) (FIG. 37), indicating that IL-11 promotes the activation of JAK pathway. To support the notion that IL-11 contributes to cisplatin resistance of ovarian cancer cells, it was found that addition of recombinant IL-11 to the medium of cisplatin sensitive cells SKOV3 significantly increased its resistance to cisplatin (FIG. 38).

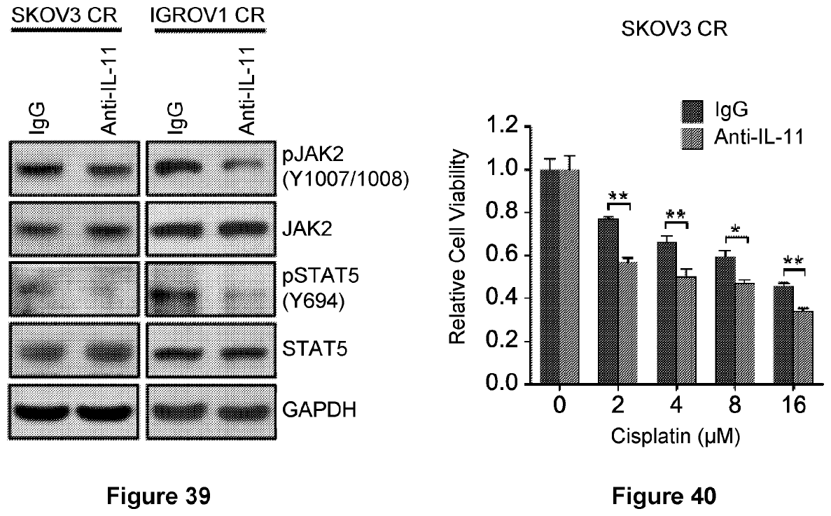

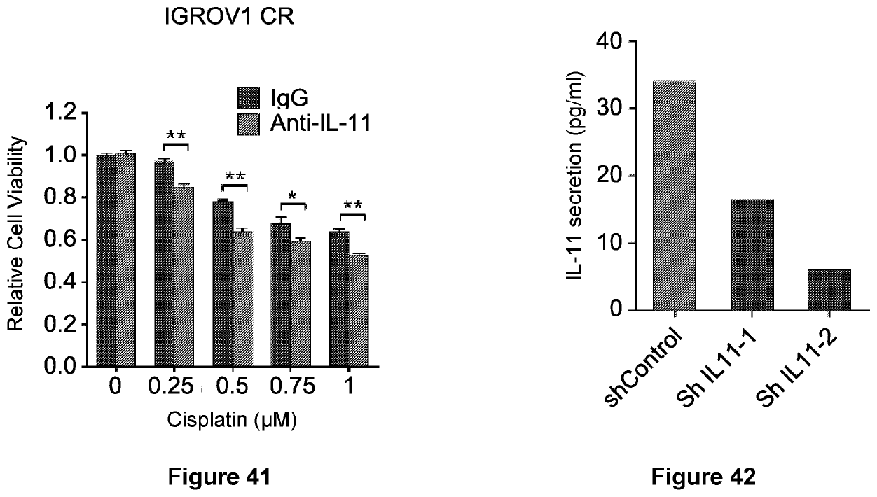

If IL-11 activates the JAK2 pathway, the addition of anti-IL-11 antibody to the medium to neutralize IL-11 may downregulate JAK2/STAT5 and re-sensitize cisplatin resistant cells to cisplatin. The addition of anti-IL-11 antibody (R&D Systems) to the medium of both SKOV3 CR and IGROV1 CR reduced the phosphorylation of JAK2 (Y1007/1008) as well as phosphorylation of STAT5 (Y694) (FIG. 39) and re-sensitized both resistant cells to cisplatin (FIGS. 40 and 41). Thus, secreted IL-11 acts as an autocrine factor to stimulate the activation of the JAK2-STAT5 pathway, thereby inducing cisplatin resistance in these cells.

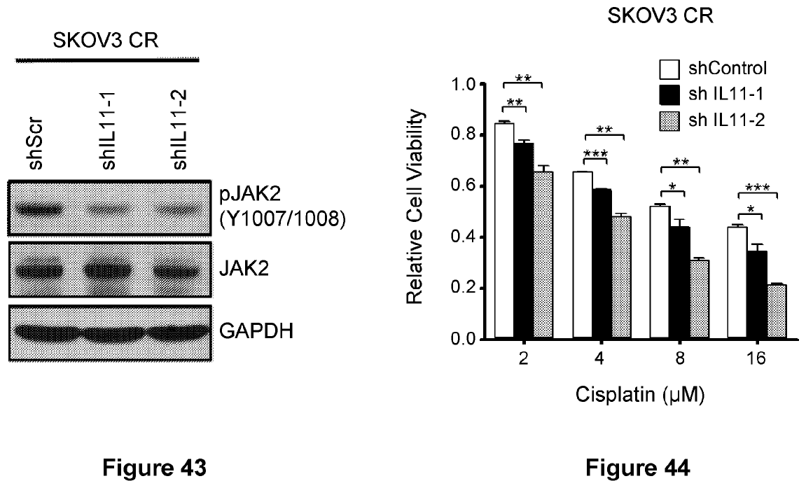

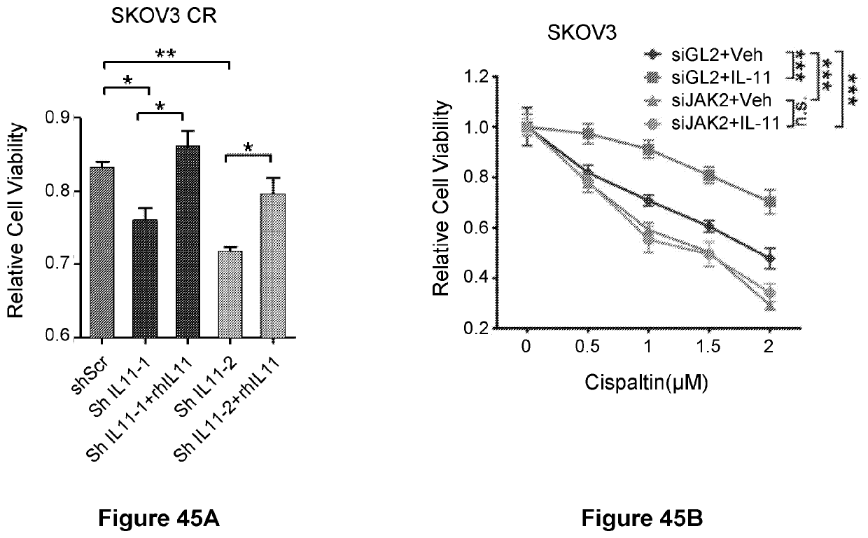

To further test the role of IL-11 in the regulation of cisplatin resistance in ovarian cancer cells, IL-11 was depleted by shRNA in SKOV3 CR cells (FIG. 42) and it was found that downregulation of IL-11 did reduce the activation of JAK2 as well as STAT5 (FIG. 43). Significantly, depletion of IL-11 was able to re-sensitize SKOV3 CR cells to cisplatin (FIG. 44), consistent with the results obtained by using anti-IL-11 antibody (FIGS. 40 and 41). Recombinant IL-11 (R&D Systems) was also added to IL-11 depletion cells to rescue. The result showed adding IL-11 back can rescue the re-sensitized shIL-11 SKOV3 CR cells (FIG. 45A). Taken together, these results demonstrate that IL-11 is up-regulated in cisplatin resistant ovarian cancer cells in vivo and in vitro, and IL-11 directly regulates the activation of JAK2 pathway and contributes to the cisplatin resistance.

To further confirm that IL-11-induced cisplatin resistance is JAK2-dependent, JAK2 was downregulated by siRNA (Santa Cruz Biotechnology) in SKOV3 cells treated with recombinant IL-11 for four hours and then cisplatin for five days (FIG. 45B). Although the addition of recombinant IL-11 could induce resistance to cisplatin in cells containing JAK2, IL-11 no longer induced cisplatin resistance in the cells with downregulated JAK2 (FIG. 45B). These findings indicate that JAK2 signaling is a major mechanism involved in IL-11-induced cisplatin resistance in SKOV3 cells.

### Example 6

### Cisplatin Treatment can Induce IL-11 Autocrine and JAK2/STAT5 Activation In Vitro and In Vivo

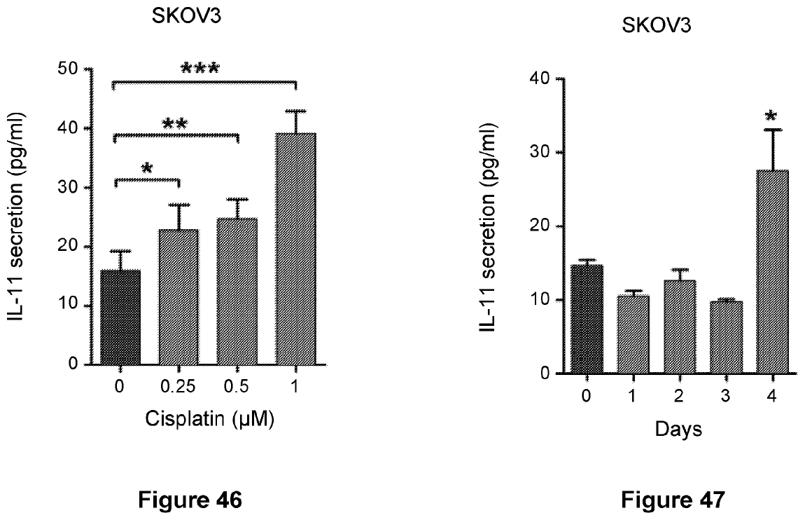

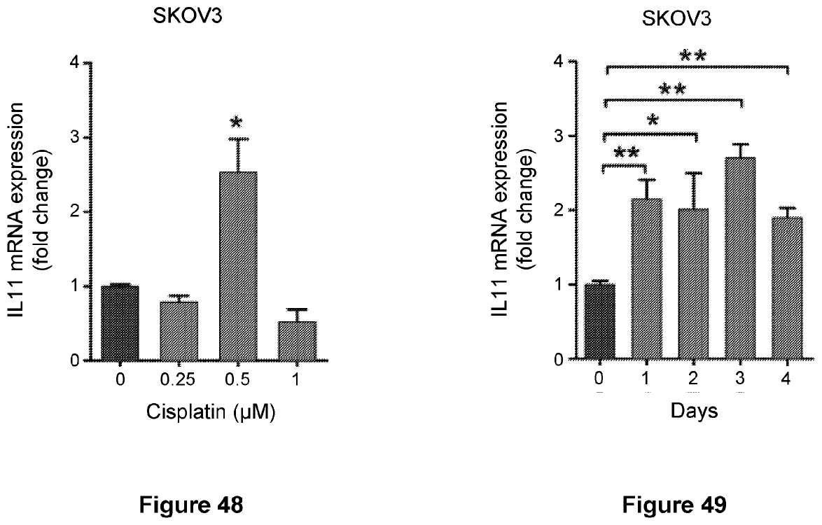

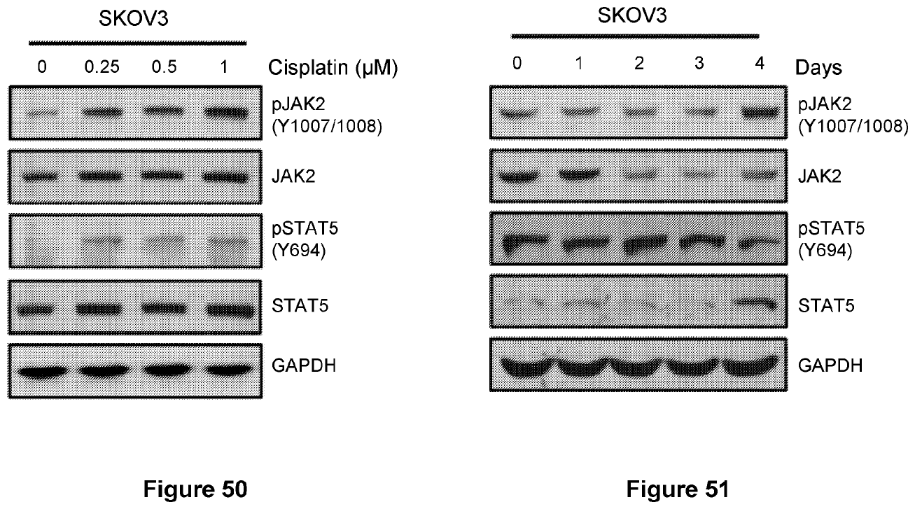

To determine whether cisplatin treatment stimulates expression of IL-11 as well as its secretion, SKOV3 cells were treated at various small doses of cisplatin for four days. It was found that IL-11 secretion was increased in a cisplatin dose dependent manner (FIG. 46). Moreover, the IL-11 secretion was examined in a time course, and increased IL-11 secretion was observed on day four post cisplatin treatment (FIG. 47). The pattern of increased secretion mirrored the upregulation of IL-11 mRNA, indicating that elevated secretion of IL-11 is due to the up-regulated transcription (FIGS. 48 and 49). Consistently, JAK2/STAT5 signaling was activated corresponding to the increased IL-11 secretion (FIGS. 50 and 51). These results suggest that IL-11 as well as the JAK pathway is up-regulated in ovarian cancer cells and this up-regulated pathway may be sustained in ovarian cancer cells after long term cisplatin treatment, resulting in resistance of these cells to cisplatin.

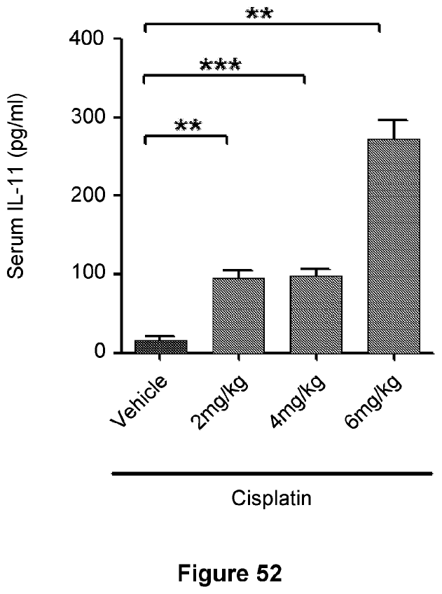

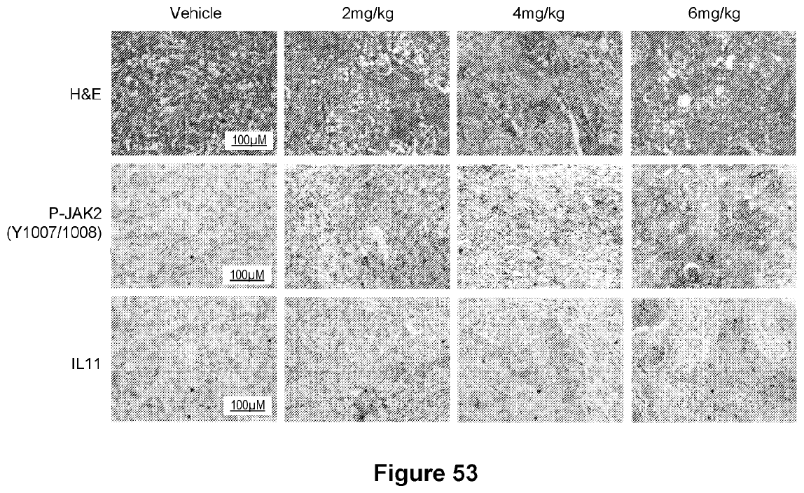

To further test whether ovarian cancer cells have a similar response to cisplatin in vivo, SKOV3 xenograft tumors were treated with various doses of cisplatin (2 mg-6 mg/kg/twice per week) for 2 weeks. Mice were sacrificed 4 days after the last treatment, and blood samples and xenograft tumors were collected to examine IL-11 expression. Significantly, cisplatin treatment dramatically increased human IL-11 levels in serum of nude mice (FIG. 52). IHC analysis indicated a significant increase of IL-11 as well as pJAK2 (Y1007/1008) levels in SKOV3 tumors. A high dose of cisplatin generated the high levels of both IL-11 and pJAK2 (Y1007/1008) on tumors (FIG. 53). Thus, cisplatin is able to promote expression of IL-11 as well as JAK2 pathway both in vitro and in vivo. These results suggest that IL-11 contributes to cisplatin resistance of ovarian cancer cells.

### Example 7

### IL-11 and JAK2 Activation Predicts Mortality of Patients Treated with Chemotherapy

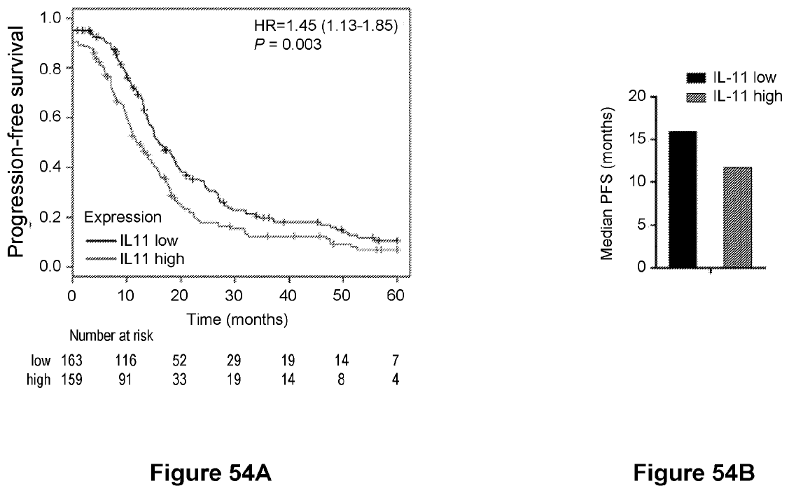

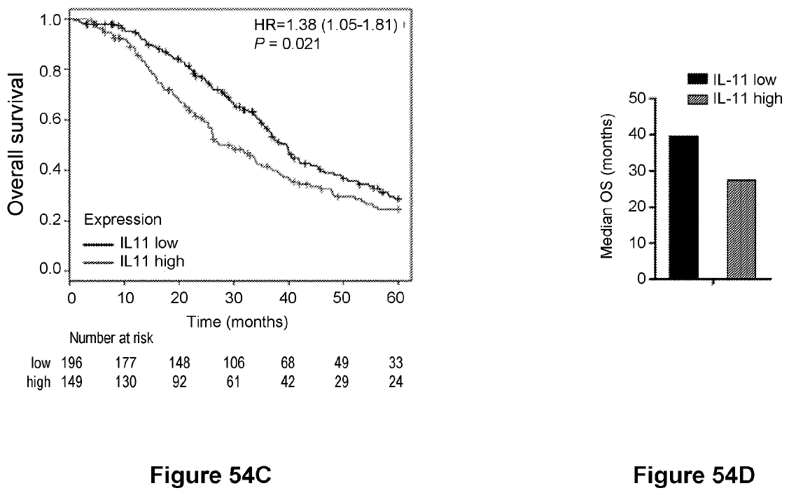

Given that the results herein show that elevated IL-11 levels contribute to cisplatin resistance of ovarian cells, and given that platinum drugs are the standard treatment for ovarian cancer patients, it was hypothesized that IL-11 levels may affect ovarian cancer patient survival rate. To test this hypothesis, Kaplan-meier plotter was used to analyze 15 databases (including TCGA) and 1816 patients. Patients with suboptimal debulk surgery (>1 cm residual disease) followed by platinum-based chemotherapy were selected. This subgroup of patients should reflect the IL-11 level and patient response to platinum based chemotherapy. The Kaplan-Meier survival analysis showed that patients with higher expression of IL-11 in their tumors had a worse 5-year progressive free survival (PFS, n=322) and 5-year overall survival (OS, n=345) than did patients with lower expression of IL-11 (FIGS. 54A and B). The median PFS was increased 4.2 months in the IL-11 low expression group to the IL-11 high expression group (15.9 months versus 11.7 months). The median OS was increased 12.3 months in the IL-11 low expression group to the IL-11 high expression group (39.57 months versus 27.27 months).

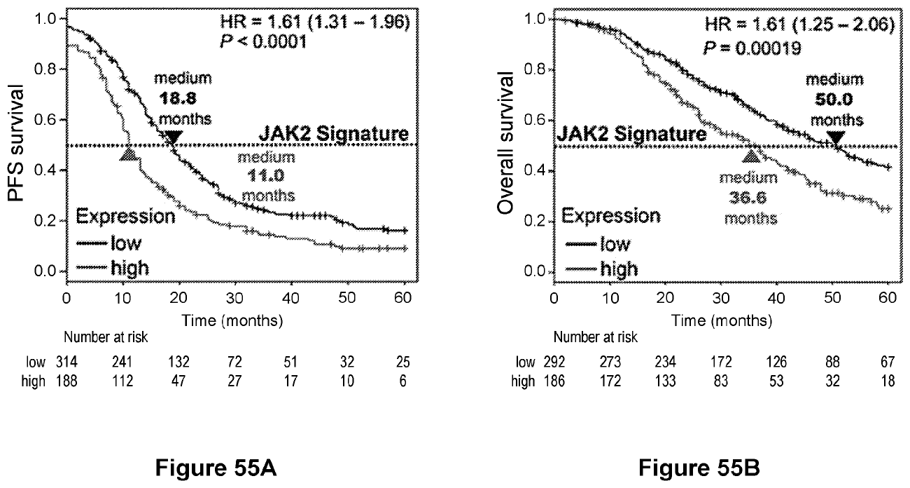

To further investigate the role of JAK2 activation in the regulation of platinum drug resistance in ovarian cancer, the correlation of JAK2 activity with ovarian patient survival rate was examined using ovarian cancer databases. Since JAK2 phosphorylation but not protein levels were determined to be up-regulated in platinum-resistant cells, the correlation of the JAK2 pathway with cisplatin resistance was investigated by analyzing and comparing the JAK2-regulated functional pathways and gene expression profiles obtained from genomic sequencing. Specifically, the genes that are functionally linked with JAK2 were analyzed by using PathwayNet, available at http://pathwaynet.princeton.edu/, and a total of 500 genes linked to JAK2 activity were identified. Analyzing gene expression from RNA-Seq data, a total of 1085 genes that are up-regulated at least 2-fold in SKOV3 CR cells as compared to SKOV3 cells were identified. Comparing these two sets of genes, a total of 22 overlapped genes were identified and were named as JAK2 signature genes. Given that JAK2 activity is up-regulated in cisplatin resistant cells, it was hypothesized that the levels of these JAK2 signature genes should inversely correlate to survival rate. These JAK2 signature genes were analyzed and compared from 1816 patients found in 15 datasets using a Kaplan-Meier plotter 32. Indeed, patients with platinum drug treatment history exhibited a worse 5-year progression free survival (PFS) and overall survival (OS) when JAK2 signature genes expression levels in their tumors were higher (FIGS. 55A and B). Thus, a higher activity of the JAK2 pathway is highly correlated with worse ovarian cancer patient survival following platinum drug-based therapy.

### Example 8

### ROS Induces Autocrine Activation of IL-11 by Promoting Expression of FOSL1 (FRA1)

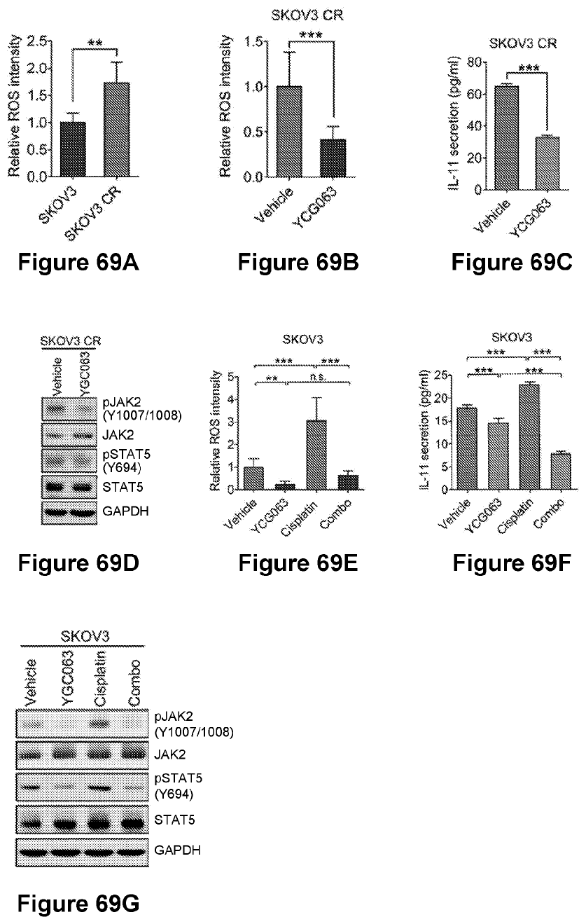

DNA damage induces generation of reactive oxygen species (ROS). Kang et al., Cell Death Dis 3: e249 (2012) and Tasdogan et al., Cell Stem Cell 19: 752-767 (2016). As such, the involvement of ROS in upregulation of IL-11 was investigated. To this end, the ROS level between SKOV3 and SKOV3 CR cells was compared. ROS was detected with a live cell-permeable, fluorophore CellROX Orange reagent (Thermo Fisher Scientific) according to the manufacturer's instructions. After treatment, cells were incubated with CellROX Orange reagent and Hoechst (Thermo Fisher Scientific) at 37° C. for 30 min, followed by washing twice with prewarmed PBS. Cells were imaged using a Nikon Eclipse 80i microscope. ROS intensity was analyzed using ImageJ (NIH) software. It was found that the basal ROS level in SKOV3 CR cells was significantly higher than that in SKOV3 cells (FIG. 69A). To investigate if ROS in SKOV3 CR cells regulate IL-11 expression, SKOV3 CR cells were treated with the ROS inhibitor YCG063 (Calbiochem), and it was found that inhibition of ROS significantly reduced IL-11 secretion as well as JAK2 and STAT5 phosphorylation (FIG. 69B-D), indicating that ROS is critical for the secretion of IL-11 in cisplatin resistant cells. Next, SKOV3 cells were treated with cisplatin, YCG063, or both. Cisplatin treatment alone activated ROS production, while YCG063 suppressed ROS production in SKOV3 cells (FIG. 69E). Furthermore, inhibition of ROS in SKOV3 cells suppressed cisplatin-induced IL-11 secretion (FIG. 69F) as well as phosphorylation of JAK2 and STAT5 (FIG. 69G)

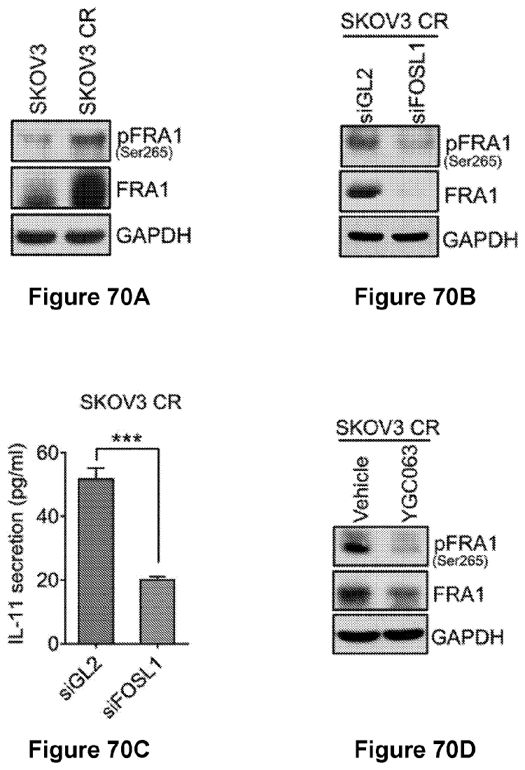

Having found that the ROS regulates IL-11 secretion, critical ROS responsive genes that regulate IL-11 expression were investigated. To achieve this goal, pathway analyses using PathwayNet were conducted, which identified a total of 20 transcription factors that likely regulate IL-11. By comparing these candidate genes with the genes identified from RNA Seq analyses, it was found that the FOSL1 was the only gene that was significantly up-regulated by more than 2-fold in SKOV3 CR cells (FIG. 70A). Further analyses indicated that the levels of FOSL1-encoded protein, FRA1, and phosphorylated (Ser265) FRA1 were increased in SKOV3 CR cells compared to SKOV3 cells (FIG. 70B). Depletion of FRA1 by FOSL1 siRNA (Thermo Fisher Scientific) significantly reduced IL-11 secretion in SKOV3 CR cells (FIGS. 70C and D), indicating that FRA1 is critical for IL-11 expression. It was next tested whether ROS is required for activation or expression of FRA1 by treating SKOV3 CR cells with YGC063. Significantly, inhibition of ROS reduced the expression of FRA1 as well as its phosphorylation (FIG. 70E), indicating that the ROS signaling is required for the activation of FRA1.

### Example 9

### Clinical Evidence of Activated IL-11-JAK2 Pathway in Platinum Drug-Resistant Ovarian Cancer Patients

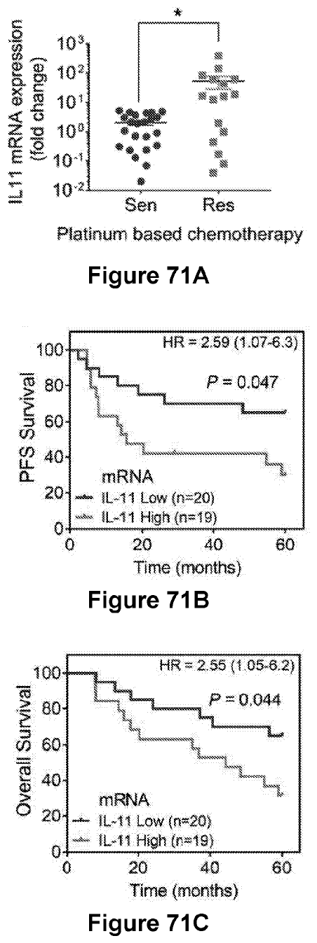

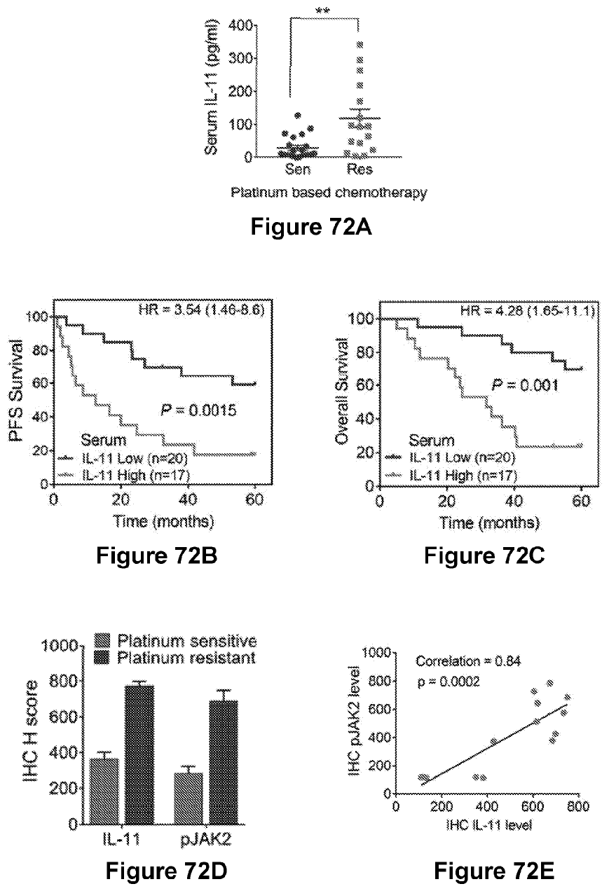

To investigate and confirm the role of the IL-11-JAK2 pathway in platinum drug resistance of ovarian cancer patients, the IL-11 mRNA levels from patient samples of a total of 23 platinum sensitive patients and 16 resistant patients were compared. It was found that IL-11 levels in the platinum drug-resistant group were higher than that in the sensitive group (FIG. 71A). Significantly, the group of patients with higher mRNA levels of IL-11 exhibited worse prognosis in terms of PFS and OS than the group with low mRNA IL11 level (FIGS. 71B and C, respectively). The serum IL-11 levels from a total of 21 platinum sensitive patients and 16 resistant patients were also examined and compared. It was found that the platinum drug-resistant group had a mean level of 120.2 pg/ml of serum IL-11, which is significantly higher than the platinum drug sensitive group (22.8 pg/ml of IL-11) (FIG. 72A). Also, the group of patients with higher serum levels of IL-11 (≥40 pg/ml) exhibited worse prognosis in terms of PFS and OS (FIGS. 72B and C, respectively) than the group with low serum IL-11 level (<40 pg/ml), indicating an inverse correlation between the serum levels of IL-11 and the survival rate of ovarian patients following platinum drug-based therapy. Out of these 37 patients, one patient was identified with both sensitive and resistant samples. The resistant tumors exhibited higher levels of IL-11 and p-JAK2 expression (FIG. 72D).

The correlation of the IL-11-JAK2 pathway in the samples from the same patient before platinum drug treatment and after tumor recurrence was also evaluated. A total of seven patients who had received platinum drug based chemotherapy and had a recurrence 11-41 months post treatment were identified. IHC staining indicated that IL-11 levels were increased in four of seven (57.1%) patients. There was a significant correlation between IL-11 and pJAK2 levels in all tested primary and recurrent patient tumor samples (FIG. 72E).

Having now fully described the methods, compounds, and compositions herein, it will be understood by those of skill in the art that the same can be performed within a wide and equivalent range of conditions, formulations, and other parameters without affecting the scope of the methods, compounds, and compositions provided herein or any embodiment thereof. All patents, patent applications and publications cited herein are fully incorporated by reference herein in their entireties as if each individual publication or patent application were specifically and individually indicated to be incorporated by reference. In addition, citation or identification of any reference in this application shall not be construed as an admission that such reference is available as prior art to the present invention.

1. A pharmaceutical composition comprising a DNA damaging agent and an inhibitor of the Janus kinase 2 (JAK2)-signal transducer and activator of transcription 5 (STAT5) pathway.

2. The pharmaceutical composition of claim 1, wherein the inhibitor is selected from the group consisting of: a JAK2 inhibitor, a STAT5 inhibitor, an interleukin-11 (IL-11) inhibitor, an IL-11 receptor (IL-11R) inhibitor, a Fos-related antigen 1 (FRA1) inhibitor, a reactive oxygen species (ROS) inhibitor, a ROS scavenger, and any combination thereof.

3. The pharmaceutical composition of claim 1, comprising a JAK2 inhibitor selected from the group consisting of: LY2784544, TG101348, TG46, and any combination thereof.

4. The pharmaceutical composition of claim 1, comprising an inhibitor selected from the group consisting of: an anti-IL-11 monoclonal antibody, an anti-IL-11R monoclonal antibody, and a combination thereof.

5. The pharmaceutical composition of claim 1, comprising a ROS inhibitor, a ROS scavenger, or a combination thereof, wherein the ROS inhibitor is YCG063 and the ROS scavenger is MnTMPyp.

6. The pharmaceutical composition of claim 1, wherein the DNA damaging agent is a platinum-based drug.

7. The pharmaceutical composition of claim 6, wherein the platinum-based drug is selected from the group consisting of: cisplatin, carboplatin, diplatinum cytostatic, iproplatin, oxaliplatin, nedaplatin, satraplatin, tetraplatin, and any combination thereof.

8. A kit comprising the pharmaceutical composition of claim 1.

9. A method of inhibiting the JAK2-STAT5 pathway in a cell, comprising administering to the cell: a) an effective dose of a DNA damaging agent; and b) an effective dose of an inhibitor of the JAK2-STAT5 pathway.

10. A method of treating cancer in a subject, comprising administering to the subject: a) an effective dose of a DNA damaging agent; and b) an effective dose of an inhibitor of the JAK2-STAT5 pathway.

11. A method of decreasing resistance to a DNA damaging agent that is used in the treatment of a disease or disorder in a subject, comprising administering to the subject: a) an effective dose of a DNA damaging agent; and b) an effective dose of an inhibitor of the JAK2-STAT5 pathway.

12. The method of claim 9, wherein the DNA damaging agent is administered prior to, concurrently with, or subsequent to the inhibitor.

13. The method of claim 9, wherein the inhibitor is selected from the group consisting of: a JAK2 inhibitor, a STAT5 inhibitor, an interleukin-11 (IL-11) inhibitor, an IL-11 receptor (IL-11R) inhibitor, a Fos-related antigen 1 (FRA1) inhibitor, a reactive oxygen species (ROS) inhibitor, a ROS scavenger, and any combination thereof.

14. The method of claim 13, comprising a JAK2 inhibitor selected from the group consisting of: LY2784544, TG101348, TG46, and any combination thereof.

15. The method of claim 13, comprising an inhibitor selected from the group consisting of: an anti-IL-11 monoclonal antibody, an anti-IL-11R monoclonal antibody, and a combination thereof.

16. The method of claim 13, comprising a ROS inhibitor, a ROS scavenger, or a combination thereof, wherein the ROS inhibitor is YCG063 and the ROS scavenger is MnTMPyp.

17. The method of claim 11, wherein the disease or disorder is a cancer.

18. The method of claim 10, wherein prior to initiation of the method the subject has been identified as having a cancer that is resistant to treatment with at least one DNA damaging agent.

19. The method of claim 10, wherein the cancer is selected from the group consisting of: ovarian cancer, testicular cancer, bladder cancer, head and neck cancer, oral cancer, esophageal cancer, lung cancer, small cell lung cancer, non-small cell lung cancer, breast cancer, cervical cancer, stomach cancer, gastric cancer, colorectal cancer, osteosarcoma, pancreatic cancer, prostate cancer, and any combination thereof.

20. (canceled)

21. (canceled)

22. The method of claim 10, wherein prior to initiation of the method the level of IL-11 mRNA or IL-11 protein, ROS, or any combination thereof in cells or blood serum in the subject is higher than in control cells or blood serum.
